SEQUENCE LISTING (2 sequences)

SEQ ID NO: 1 - protein, 393 aa, Saccharomyces cerevisiae
MSGFNFGGTGAPTGGFTFGTAKTATTTPATGFSFSTSGTGGFNFGAPFQPATSTPSTGLFSLATQTPATQTTGFTFGTATLASGGTGFSLGIGASKLNLSNTAATPAMANPSGFGLGSSNLTNAISSTVTSSQGTAPTGFVFGPSTTSVAPATTSGGFSFTGGSTAQPSGFNIGSAGNSAQPTAPATLPFTPATPAATTAGATQPAAPTPTATITSTGPSLFASIATAPTSSATTGLSLCTPVTTAGAPTAGTQGFSLKAPGAASGTSTTTSTAATATATTTTSSSTTGFALNLKPLAPAGIPSNTAAAVTAPPGPGAAAGAAASSAMTYAQLESLINKWSLELEDQERHFLQQATQVNAWDRTLIENGEKITSLHREVEKVKLDQKRLDQEL

SEQ ID NO: 2 - protein, 187 aa, Saccharomyces cerevisiae
LINKWSLELEDQERHFLQQATQVNAWDRTLIENGEKITSLHREVEKVKLDQKRLDQELDFILSQQKELEDLLSPLEELVKEQRATIYLQHADEERQKTYKLAENIDAQLKRMAQDLKDIIEHLNTSGAPADTSDPLQQICKILNAHMDSLQWIDQNSALLQRKVEEVTKVCVGRRKEQERSFRITFD

(19) United States
(12) Patent Application Publication
(10) Pub. No.: US 2006/0035823 A1
(43) Pub. Date: Feb. 16, 2006

(54) Isolated fragments of p62 nucleoporin and uses thereof

(76) Inventors: Seth Lederman, New York, NY (US); Christopher Gamper, Baltimore, MD (US)

Correspondence Address: Bryan Cave LLP, 1290 Avenue of the Americas, 33rd Floor, New York, NY, 10104, US

(21) Appl. No.: 09/930,864

(22) Filed: Aug. 16, 2001

Related U.S. Application Data
(60) Provisional application No. 60/225,938, filed on Aug. 17, 2000.

(51) Int. Cl. A61K 38/17; C07K 14/705

(52) U.S. Cl. 514/12; 530/350

(57) ABSTRACT

The invention is directed to isolated protein fragments of p62 nucleoporin including deletion isoforms and nucleic acid sequences encoding these deletion isoforms. The isolated deletion isoforms disclosed herein include the sequences: SEQ. ID NO.:1 MSGFNFGGTG APTGGFTFGT AKTATTTPAT GFSFSTSGTG GFNFGAPFQP ATSTPSTGLF SLATQTPATQ TTGFTFGTAT LASGGTGFSL GIGASKLNLS NTAATPAMAN PSGFGLGSSN LTNAISSTVT SSQGTAPTGF VFGPSTTSVA PATTSGGFSF TGGSTAQPSG FNIGSAGNSA QPTAPATLPF TPATPAATTA GATQPAAPTP TATITSTGPS LFASIATAPT SSATTGLSLC TPVTTAGAPT AGTQGFSLKA PGAASGTSTT TSTAATATAT TTTSSSTTGF ALNLKPLAPA GIPSNTAAAV TAPPGPGAAA GAAASSAMTY AQLESLINKW SLELEDQERH FLQQATQVNA WDRTLIENGE KITSLHREVE KVKLDQKRLD QEL; SEQ ID NO.:2 LINKWSLELE DQERHFLQQA TQVNAWDRTL IENGEKITSL HREVEKVKLD QKRLDQELDF ILSQQKELED LLSPLEELVK EQRATIYLQH ADEERQKTYK LAENIDAQLK RMAQDLKDII EHLNTSGAPA DTSDPLQQIC KILNAHMDSL QWIDQNSALL QRKVEEVTKV CVGRRKEQER SFRITFD. The invention is also directed to peptides which are at least 80% identical over their entire amino acid sequence set forth in SEQ ID NO:1, and SEQ ID NO:2 and salts thereof. Pharmaceutical compositions including the polypeptides, their isoforms, and methods for their use activating target genes are also provided.

This application claims the benefit of U.S. Provisional Application No. 60/225,938, filed Aug. 17, 2000.

### GOVERNMENT GRANTS

The invention disclosed herein was made with Government support under NIH/NCI Grant No. RO-1-CA55713 from the Department of Health and Human Services. Accordingly, the U.S. Government has certain rights in this invention. In addition, the invention disclosed herein was made with assistance from personnel supported by the Medical Scientist Training program Grant No. 5-T32-GM07367.

Throughout this application, various publications are referred to within parentheses. Disclosure of these publications in their entireties are hereby incorporated by reference into this applicaation to more fully describe the state of the art to which this invention pertains.

### BACKGROUND OF THE INVENTION

TRAF-3 gene products are signaling molecules that interact with the cytoplasmic tails of CD40 (Cheng, et al., 1995; Hu, et al., 1994; Sato, et al., 1995), other TNF-R family members (Mosialos, et al., 1995; Gedrich, et al., 1996; Boucher, et al., 1997; Yamamoto, et al., 1998; Vanarsdale, et al., 1997; Arch and Thompson, 1998; Kawamata, et al., 1998) and the Epstein-Barr virus latent membrane protein, LMP1 (Mosialos, et al., 1995). TRAF-3 is essential for T cell-dependent antibody production (Xu, et al., 1996) and TRAF-3 splice-deletion isoforms activate NF-6B (van Eyndhoven, et al., 1999), which is known to be important in this process (Berberich, et al., 1994; Snapper, et al., 1996; Hostager, et al., 1996; Hsing and Bishop, 1999; Grumont, et al., 1998; Attar, et al., 1998; Horwitz, et al., 1999). However, the mechanisms by which TRAF-3 mediates signaling are not completely understood. Since TRAF-3 is a cytoplasmic, not a nuclear protein (Mosialos, et al., 1995) and appears to lack catalytic activity, as do the cytoplasmic tails of all known mammalian TNF-R family members (Becraft, et al., 1996), it is generally believed that TRAF-3 is an adapter molecule. In this regard, TRAF-3 has been found to interact with several other molecules including NF-6B inducing kinase (NIK) (Song, et al., 1997; Malinin, et al., 1997), Apoptosis Signal-regulating Kinase 1 (ASK1) (Nishitoh, et al., 1998), TRAF-5 (Pullen, et al., 1998) and I-TRAF/TANK (Rothe, et al., 1996; Cheng and Baltimore, 1996). It has been suggested that such interactions provide the means by which TRAF-3 mediates signal transduction, but it is unknown whether TRAF-3 associates with other molecules in an active signaling complex and/or shuttles between the inner surface of the cell membrane and some other subcellular location.

TRAF-3 is known to serve essential, non-redundant functions, since TRAF-3 deficient mice are runted and die shortly after birth (Xu, et al., 1996). Furthermore, TRAF-3 plays an essential and non-redundant role in signaling events underlying T cell-directed B cell differentiation, since lethally irradiated mice reconstituted with TRAF-3^{-/-} lymphocytes exhibit defective T-dependent antibody formation (Xu, et al., 1996). NF-6B activation is relevant to this process because inhibition of NF-6B activation significantly impairs T-dependent antibody formation in vivo (Snapper, et al., 1996; Hostager, et al., 1996; Hsing and Bishop, 1999; Grumont, et al., 1998; Attar, et al., 1998; Horwitz, et al., 1999). In addition, over-expression of certain TRAF-3 splice-deletion isoforms, induces NF-6B activation (van Eyndhoven, et al., 1999). T-dependent antibody production is also known to depend on CD40 signaling (Lederman, et al., 1996), which induces NF-6B activation (Berberich, et al., 1994). Together, these findings suggest that TRAF-3 mediated NF-6B activation plays a role in T-dependent antibody production.

Full-length TRAF-3 contains five putative protein domains termed (from C- to N-terminus): the TRAF-C, TRAF-N, isoleucine zipper, Zn finger and Ring finger domains (Cheng, et al., 1995; Hu, et al., 1994; Sato, et al., 1995; Mosialos, et al., 1995). Some functional roles have been associated with particular domains. The TRAF-3/TRAF-C domain, a structural homology element which defines TRAF family members, is known to be important for the interaction of TRAF-3 with the cytoplasmic tails of TNF-R family receptors (Cheng, et al., 1995; Force, et al., 1997; Vanarsdale, et al., 1997) binding to cytoplasmic proteins such as I-TRAF/TANK (Rothe, et al., 1996; Cheng and Baltimore, 1996) and NIK (Regnier, et al., 1997), and homo-oligomerization (Cheng, et al., 1995; Force, et al., 1997; Sato, et al., 1995; Pullen, et al., 1998; Malinin et al., 1997). Together, the TRAF-N and TRAF-C domains are believed to allow formation of TRAF-3 homo-trimers, since critical residues involved in TRAF-2 homo-trimer formation are conserved in TRAF-3 (Park, et al., 1999).

The TRAF-3 isoleucine zipper (or coiled-coil) domain has been shown to participate in TRAF-3 interactions with TRAF-5, the only other known TRAF that contains an isoleucine zipper (Pullen, et al., 1998). The TRAF-3 Zn finger domain contains five atypical Zn fingers in full-length TRAF-3 and fewer fingers with different compositions in splice-deletion isoforms that induce NF-6B activation (Eyndhoven, et al., 1999). The TRAF-3 Zn fingers and Ring finger are required for the ability to induce NF-6B activation in both TRAF-3 splice-variants and in TRAF-3/TRAF-5 chimeric molecules (Dadgostar and Cheng, 1988). However, the interactions between the TRAF-3 Ring and Zn fingers with other factors that regulate NF-6B activation and translocation are not completely understood. The mechanism by which TRAF-3 gene products induce NF-6B activation between receptor stimulation and translocation of activated NF-6B complexes into the nucleus remain unclear.

NF-6B proteins are transcription factors that form homo- and hetero-dimeric complexes which are retained in the cytoplasm bound to I6B proteins in resting cells (reviewed in (Baeuerle and Baltimore, 1996)). Certain stimuli, such as signaling by several TNF-R family members, activate the I6B (IKK) complex which phosphorylates I6B proteins, ultimately releasing free NF-6B dimers with exposed nuclear localization sequences (NLSs) (DiDonato, et al., 1997; Woronicz, et al., 1997; Mercurio, et al., 1997). Like other NLS containing molecules, the freed NF-6B complexes associate with the karyopherin-∀ (importin-∀) cytoplasmic NLS receptor (Nadler, et al., 1997). The karyopherin-∀/NF-6B complex is targeted to the nuclear pore by association with karyopherin-∃ (Gorlich, et al., 1995) which mediates interaction of the complex with p62 nucleoporin (p62) (Rexach and Blobel, 1995; Finlay, et al., 1991; Percipalle, et al., 1997). The p62 C-terminal domain binds karyopherin-∃ (Percipalle, et al., 1997) and an N-terminal domain binds p10/NTF2, an accessory factor that is required for the docked import complex to undergo translocation through the pore (Paschal and Gerace, 1995; Torgerson, et al., 1998). After translocation, NF-6B complexes bind to genomic regulatory sequences and activate transcription of target genes.

Although it was known that NF-6B activation and translocation across a nuclear membrane were relevant to the role TRAF-3 plays in signaling events underlying T cell-directed B cell differentiation, it was not known, expected or realized that particular fragments of the p62 nucleoporin polypeptide effect this process in ways that the complete p62 polypeptide does not. Thus, the polypeptides of the present invention regulate T cell-dependent antibody production against antigens and provide new immunotherapies and treatments.

### SUMMARY OF THE INVENTION

The present invention relates to polypeptide sequences derived from the p62 nucleoporin which display distinct biological activity. The present invention also relates to polynucleotide sequences that encode these fragments.

One embodiment of the present invention provides an isolated polypeptide derived from the p62 nucleoporin protein, p62(1-392), of the structure of formula I and salts thereof:

MSGFNFGGTG APTGGFTFGT AKTATTTPAT GFSFSTSGTG GFNFGAPFQP ATSTPSTGLF
SLATQTPATQ TTGFTFGTAT LASGGTGFSL GIGASKLNLS NTAATPAMAN PSGFGLGSSN LTNAISSTVT
SSQGTAPTGF VFGPSTTSVA PATTSGGFSF TGGSTAQPSG FNIGSAGNSA QPTAPATLPF TPATPAATTA
GATQPAAPTP TATITSTGPS LFASIATAPT SSATTGLSLC TPVTTAGAPT AGTQGFSLKA PGAASGTSTT
TSTAATATAT TTTSSSTTGF ALNLKPLAPA GIPSNTAAAV TAPPGPGAAA GAAASSAMTY AQLESLINKW
SLELEDQERH FLQQATQVNA WDRTLIENGE KITSLHREVE KVKLDQKRLD QEL.

In another embodiment of the invention, isolated polypeptides having amino acid sequences that are at least about 80% identical over their entire length to the amino acid sequence set forth in formula I, and salts thereof, are provided.

The present invention also provides an isolated polypeptide derived from the p62 nucleoporin, p62(336-522), of the structure of formula II and salts thereof:

LINKWSLELE DQERHFLQQA TQVNAWDRTL IENGEKITSL HREVEKVKLD QKRLDQELDF
ILSQQKELED LLSPLEELVK EQRATIYLQH ADEERQKTYK LAENIDAQLK RMAQDLKDII EHLNTSGAPA
DTSDPLQQIC KILNAHMDSL QWIDQNSALL QRKVEEVTKV CVGRRKEQER SFRITFD.

Isolated polypeptides having amino acid sequences that are at least about 80% identical over their entire length to the amino acid sequence set forth in formula II and salts thereof, are also provided.

The invention also provides pharmaceutical compositions comprising a therapeutically effective amount of a polypeptide of the invention and a pharmaceutically acceptable carrier. Methods of inhibiting translocation of activated NF-6B and enhancing activation of NF-6B in a mammal by administering a pharmaceutical composition of the invention, is also provided. The invention further provides methods of providing p62(1-392) or p62(336-522) to cells comprising contacting polypeptides with cells that have been treated to absorb exogenous polypeptides so that the cells contain the polypeptide of the invention.

Another embodiment of the invention provides methods of screening a plurality of test compounds for an effect on a biochemical system. The method of screening compounds comprises incubating components of a biochemical system, at least one test compound and at least one of the polypeptides selected from the group consisting of a polypeptide having the amino acid sequence set forth in SEQ ID NO:1, a polypeptide having an amino acid sequence that is at least 80% identical over its entire length to a polypeptide having the amino acid sequence set forth in SEQ ID NO:1, a polypeptide having the amino acid sequence set forth in SEQ ID NO:2, and a polypeptide having an amino acid sequence that is at least 80% identical over its entire length to a polypeptide having the amino acid sequence set forth in SEQ ID NO:2. The method also includes a step designed for detecting an effect of the test compound on interactions between the components of the biochemical system.

### BRIEF DESCRIPTION OF THE DRAWINGS

FIG. 1 illustrates a yeast two-hybrid analysis of TRAF-3 protein interactions with p62 nucleoporin. Yeast two-hybrid analysis of p62 interactions with TRAF-2, -3, -4, -5, and -6 are shown in portion B of FIG. 1. Here, the yeast were transfected with empty AD vector or AD/p62(336-522) vector and the indicated BD/TRAF protein expression vector.

FIG. 2 is a summary of the p62 nucleoporin constructs obtained in the library screen (black bars) or generated by subcloning (grey bars) and their interactions with full-length TRAF-3 in the yeast two-hybrid system. Colonies that grew in the absence of His and were blue by colony lift ∃-gal assay are “positive” and colonies that failed to grow or develop blue color are “negative”. Portion B of FIG. 2 illustrates a diagrammatic summary of TRAF-3 deletion constructs tested for interactions with p62(336-522) in the yeast two-hybrid system.

FIG. 3 illustrates the results of an analysis of TRAF-3:p62 association in 293T cells. Cell lysates from 293T cells transiently transfected with the indicated epitope-tagged expression vectors were immunoprecipitated with either anti-X-press or anti-HA monoclonal antibodies. Detected proteins are labeled at the left and IgG heavy and light chains are labeled at the right. The asterix indicates a proteolytic fragment of His-TRAF-3 which was observed when p62(336-522) was co-expressed.

FIG. 4 illustrates the results of the effects of TRAF-3 and p62 on RelA(RelA) induced NF-6B activation in mammalian cells. The white bars represent the activity of cultures co-transfected with empty expression vector, while grey bars represent the activity of cultures co-transfected with 100 ng of pCDNA3/p65(RelA). Empty pCEP4 was used to normalize DNA content of all samples. Renilla luciferase activity was used to scale Firefly luciferase activity for transfection efficiency. Results are normalized to the activity of cultures transfected with empty pCEP4 without RelA co-transfection. Error bars represent standard deviation of the mean of triplicate cultures. These data are representative of 3 independent experiments.

FIG. 5 illustrates the results of the effects of TRAF-3 and p62 on CD40 induced NF-6B activation in 293T cells. 293T cells were transiently transfected with 3 ug of the indicated expression vectors, 300 ng of the PRDIIx4 Luc NF-6B reporter construct and 75 ng of pRLtk. White bars represent the activity of cultures co-transfected with empty expression vector and grey bars indicate the activity of cultures co-transfected with 500 ng of pCEP4/CD40 to activate NF-6B. Empty pCEP4 was used to normalize DNA content of all samples. Renilla luciferase activity was used to scale Firefly luciferase activity for transfection efficiency. Results are normalized to the activity of cultures transfected with empty pCEP4 without CD40 co-transfection. Error bars represent standard deviation of the mean of triplicate cultures. These data are representative of 3 independent experiments.

FIG. 6 illustrates RNAse protection analysis of TRAF-3 mRNA splice variant expression. A, A schematic representation of the TRAF-3 genomic sequence, with exons represented as wide black bars, is mapped onto the protein domains encoded by full-length TRAF-3 cDNA. Below are depicted the protected probe fragments resulting from hybridization of an anti-sense probe corresponding to the full-length TRAF-3 mRNA with all previously identified alternative splice forms of TRAF-3 mRNA. As indicated, most “secondary” protected fragments, may have contributions from multiple splice-variants. B, RNAse protection of the indicated samples was performed using an antisense human GAPDH probe as described in Materials and Methods. The indicated band (absent from the yeast tRNA sample) corresponds to the expected 316 nt protected fragment resulting from hybridization of the probe with GAPDH mRNA. C, TRAF-3 splice variant RNAse protection assay for Yeast tRNA (negative control), Ramos CC total RNA (negative control), and Jurkat D1.1 total RNA (positive control) using indicated probes. (Full-length TRAF-3 probe is indicated by “FL”.) D, TRAF-3 splice variant RNAse protection assay for BJAB, Daudi, and Raji total RNA using the indicated probes. In C and D, diluted probes were run as size markers at the left and right of each gel. Displayed below each gel is the signal from each sample resulting from hybridization of probes to the internal control sense TRAF-3 RNA. Primary fragments corresponding to hybridization of a given probe with its complementary mRNA splice-variant are indicated by an asterisk (*). The asterisk in each Δ130 lane marks the expected position for the Δ130 primary fragment. An unexpected TRAF-3 specific band with an approximate size of 165 nt was protected by the probes FL, Δ25, Δ27, Δ52, Δ56, and Δ83 and is indicated by a cross (†) Data shown are representative of three independent experiments.

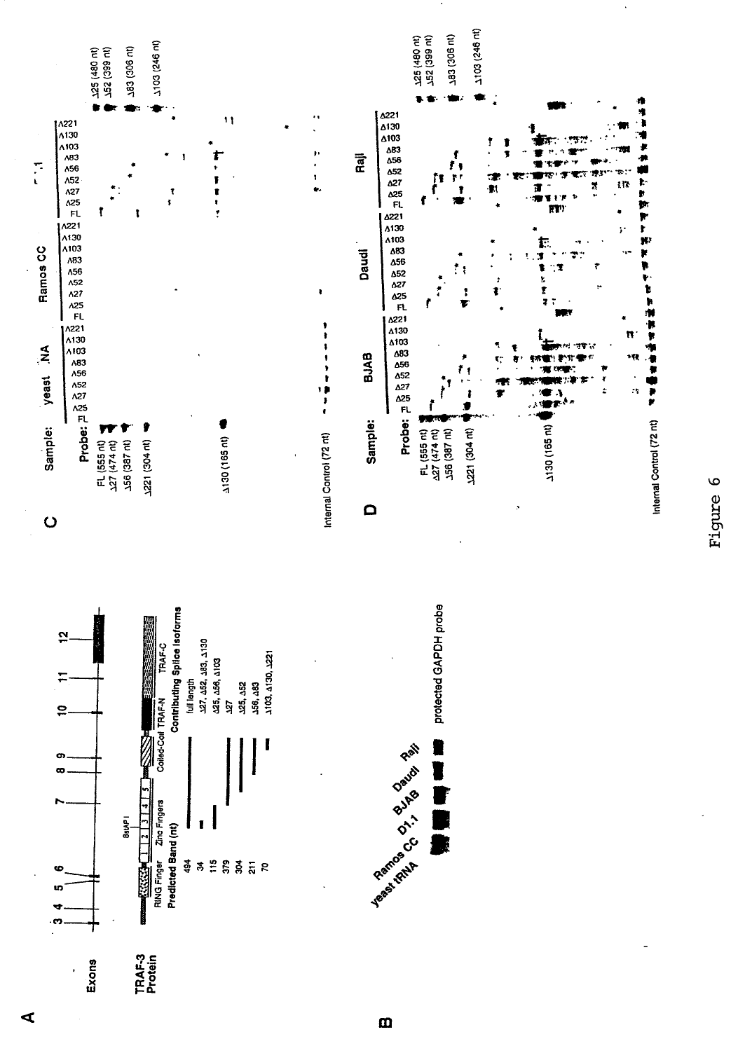

FIG. 7 illustrates relative expression of TRAF-3 splice isoforms. Phosphorimaging analysis of the RNAse protection assay shown in FIG. 1 as described in Materials and Methods. Data are presented as arbitrary units of TRAF-3 expression, scaled to the expression of full length TRAF-3 in Jurkat D1.1 as 100.

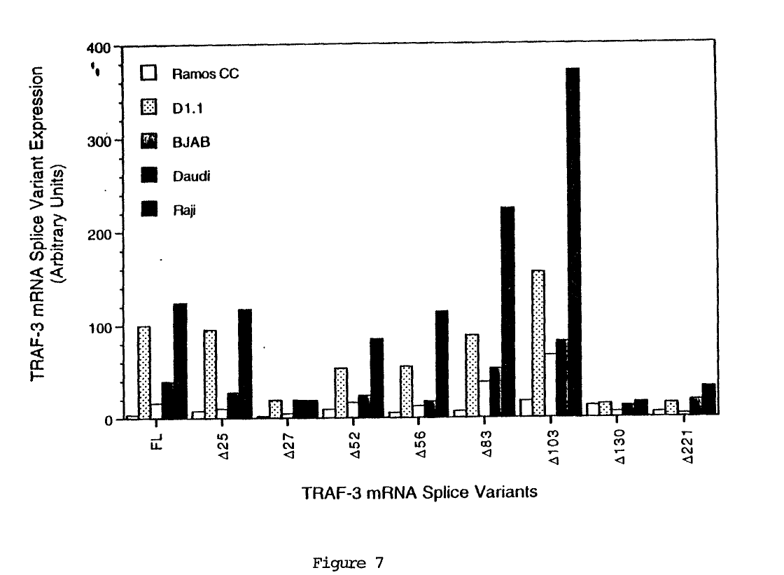

FIG. 8 illustrates effect of TRAF-3 splice-variants on NF-κB signaling in BJAB cells. BJAB cells were transiently transfected with the indicated expression vectors by electroporation. Thirty-six hours after transfection, cells were harvested and NF-κB dependent luciferase activity was measured. Data are the mean of triplicate samples, scaled to signals from control, LacZ transfected cells. Error bars represent standard deviation. This experiment is representative of three independent experiments.

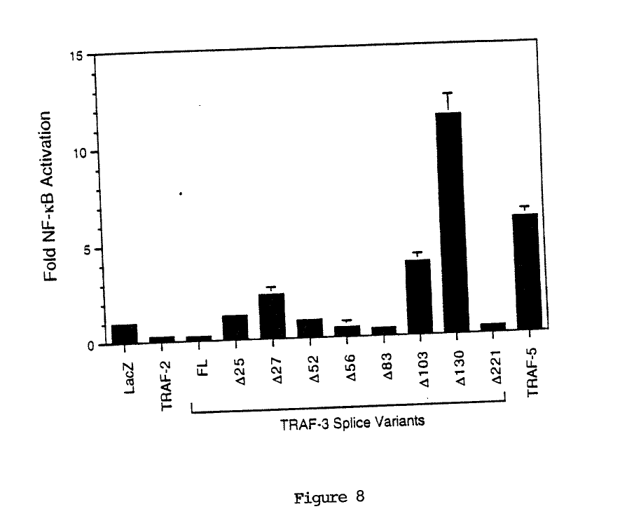

### DETAILED DESCRIPTION OF THE INVENTION

Abbreviations Used Throughout Application

Throughout this application various abbreviations are used. These abbreviations and what they refer to are listed below.

TRAF, TNF receptor-associated factor; NF-6B, Nuclear factor-6B; TNF-R, Tumor Necrosis Factor Receptor; NIK, NF-6B-including kinase; I-6B, Inhibitor of NF-6B; IKK, I-6B Kinase; N-terminal; amino-terminal; C-terminal, carboxy-terminal; p62, p62 nucleoporin; NLS, nuclear localization sequence; ORF, open reading frame; RT, reverse transcriptase; AD, Activation Domain; BD, DNA Binding Domain, ∃-gal, ∃-galactosidase; His, histidine; aa, amino acid; HA-tagged, hemagglutinin epitope tagged fusion construct; TBST, Tris-buffered saline with 0.1% Tween-20; IMDM Iscove's Modified Dulbecco's Media.

Single and Three Letter Abbreviations for Amino Acids

| | | |
|---|---|---|
| A = | Ala = | Alanine |
| R = | Arg = | Arginine |
| N = | Asn = | Asparagine |
| D = | Asp = | Aspartic Acid |
| B = | Asx = | Asparagine or aspartic acid |
| C = | Cys = | Cysteine |
| Q = | Gln = | Glutamine |
| E = | Glu = | Glutamic Acid |
| Z = | Glx = | Glutamine or Glutamic Acid |
| G = | Gly = | Glycine |
| H = | His = | Histidine |
| I = | Ile = | Isoleucine |
| L = | Leu = | Leucine |
| K = | Lys = | Lysine |
| M = | Met = | Methionine |
| F = | Phe = | Phenyalanine |
| P = | Pro = | Proline |
| S = | Ser = | Serine |
| T = | Thr = | Threonine |
| W = | Trp = | Tryptophan |
| Y = | Tyr = | Tyrosine |
| V = | Val = | Valine |

It shall be understood that the term “polypeptide” as used herein, refers to a chain of amino acids linearly linked together by peptide bonds.

It shall be also understood that the phrases “nucleic acid” or “nucleic acid sequence” as used herein, refer to an oligonucleotide, nucleotide sequence, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, the term “fragments” refers to those nucleic acid sequences which are greater than about 60 nucleotides in length.

It shall also be understood that the terms “operably associated” or operably linked,” as used herein, refer to functionally related nucleic acid sequences. A promoter is operably associated or operably linked with a coding sequence if the promoter controls the transcription of the encoded polypeptide. While operably associated or operably linked polynucleotides can be contiguous and in reading frame, certain genetic elements, e.g., repressor genes, are not contiguously linked to the encoded polypeptide but still bind to operator sequences that control expression of the polypeptide.

The phases “NF-6B activation” or “activated NF-6B” as used herein, refer to a NF-6B molecule that dissociates from I6B, which is a complex that masks the NF-6B NLS. Signaling by cell surface receptors leads to the dissociation of I6B and the liberation of NF-6B with an exposed NLS. It is this form of NF-6B, having an exposed NLS, that is referred to herein as the activated form of NF-6B.

A “substitution,” as used herein, refers to the replacement of one or more amino acids or nucleotides by different amino acids or nucleotides, respectively. The words “insertion” or “addition,” as used herein, refer to changes in amino acid sequence resulting in the addition of one or more amino acid residues, respectively, to the sequence found in the naturally occurring molecule. The term “cDNA” as used herein, stands for complementary DNA. cDNA is complementary to a mRNA strand and is produced by extracting the mRNA (or purified subfraction of mRNA) from cells and making a complementary copy of the mRNA using the reverse transcriptase enzyme of retroviruses, which synthesizes a DNA strand from the mRNA OF template. This cDNA strand can then be converted into a double stranded molecule. The phrase “genomic DNA” is DNA that contains both introns (the coding region of DNA) and extrons (the non-coding region of DNA). Genomic DNA can be produced by cleaving the entire genome of a cell with a specific restriction nuclease to produce smaller fragments that are separated by standard separation techniques including SDS-gel.

“Transformation,” as defined herein, describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofections, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

As used herein, the phrase “biochemical system” generally refers to a chemical interaction that involves molecules of the type generally found within living organisms. Such interaction include full range of catabolic and anabolic reactions which occur in living systems including enzymatic, hinding, signaling and other reactions. Further, biochemical systems, as defined herein, will also include model systems which are mimetic of a particular biochemical interaction. Examples of biochemical systems of particular interest in practicing the present invention include, e.g., receptor-ligand interactions, enzyme-substrate interactions, cellular signaling pathways, transport reactions involving model barrier systems (e.g., nuclear membranes or membrane fractions) for bioavailability screening, and a variety of other general systems. Cellular or organismal viability or activity may also be screened using the methods and apparatuses of the present invention, i.e., in toxicology studies.

As used herein the term “receptor” generally refers to one member of a pair of compounds which specifically recognize and bind to each other. The other member of the pair is termed a “ligand.” Thus, a receptor/ligand pair may include a typical protein receptor, usually membrane associated, and its natural ligand, e.g., another protein or small molecule. Receptor/ligand pairs may also include antibody/antigen binding pairs, complimentary nucleic acids, nucleic acid associated proteins and their nucleic acid ligands. A large number of specifically associating biochemical compounds are well known in the art and can be utilized in practicing the present invention.

As used herein, the term “test compound” refers to the collection of compounds that are to be screened for their ability to affect a particular biochemical system. Test compounds may include a wide variety of different compounds, including chemical compounds, mixtures of chemical compounds, e.g., polysaccharides, small organic or inorganic molecules, biological macromolecules, e.g., peptides, proteins, nucleic acids, or an extract made from biological materials such as bacteria, plants, fungi, or animal cells or tissues, naturally occurring or synthetic compositions. Depending upon the particular embodiment being practiced, the test compounds may be provided, e.g., injected free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, glass beads, polyaminemethylvinylether analegic acid copolymer, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

In addition, throughout this application, various references are referred to in the text within parentheses in full or within parentheses by number. Disclosures of these publications in their entireties are hereby incorporated by reference into this application to more fully describe the state of the art to which this invention pertains. Full bibliographic citation for those references referred to by number may be found at the end of the application, preceding the claims.

In one embodiment, the present invention provides polypeptides derived from the p62 nucleoporin protein that inhibit the translocation of NF-6B in ways that the complete p62 nucleoporin protein does not. In particular, this invention provides for an isolated polypeptide derived from the p62 nucleoporin protein, p62(1-392), that inhibits the translocation of activated NF-6B across a nucleic membrane. The amino acid sequence of p62(1-392) is set forth as formula I in SEQ ID NO:1.

There are several domains of the p62 protein, each of which have different structures and sometimes different functions. For example, the p62 C-terminal coiled-coil domain and the TRAF-3 Zn finger and coiled-coil domains are important in mediating their interaction. The interaction of p62 with TRAF-3 is specific, since p62 does not interact with TRAF-2, -4, -5 or -6. Over expression of p62(1-392), which contains the p10/NT2 binding domain but not the TRAF-3 binding domain, inhibits RelA-induced reporter activity, and inhibits NF-6B translocation. Inhibiting NF-6B translocation prevents NF-6B from activating the gene normally expressed when NF-6B binds to a regulatory region of the target gene. Thus, inhibiting NF-6B translocation, in affect, limits, if not completely blocks the transcription of genes that are normally expressed by the binding of NF-6B to a regulatory region of a gene.

Another embodiment of the present invention provides isolated polypeptides derived from the p62 nucleoporin protein that enhance the activation of NF-6B in ways that the complete p62 nucleoporin protein does not. In particular, this invention provides for an isolated polypeptide derived from the p62 nucleoporin protein, p62(336-522), that enhances the activation of NF-6B without affecting translocation of activated NF-6B. For example, over expression of p62(336-522), a TRAF-3 binding fragment, enhances NF-6B activation and augments CD40induced NF-6B reporter gene activity, but has no effect on RelA-induced reporter activity. This indicates that the p62(336-522) enhances the activation of NF-6B and does not affect the translocation of activated NF-6B. The amino acid sequence of p62(336-522) is set forth as formula II in SEQ ID NO:2.

The invention also encompasses “variants” of the polypeptides having the sequences set forth in SEQ ID NO:1 and SEQ ID NO:2. In particular, such variant polypeptides will have at least about 80%, more preferably at least about 85%, and most preferably at least about 90% polypeptide identity over their entire length to a polypeptide set forth in either SEQ ID NO:1 or SEQ ID NO:2. In other words, a polypeptide that is 80% identical to SEQ ID NO:1 may have up to about 79 amino acid additions, deletions, substitutions or insertions with respect to SEQ ID NO:1. These structural alterations may occur at the amino acid terminus, or at the carboxyl terminus, or anywhere between termini. They may be interspersed either individually among the amino acid sequence of the compound or in one or more contiguous groups within the compound. As for SEQ ID NO:2, a polypeptide that is 80% identical to this sequence may have up to about 38 amino acid additions, deletions, substitutions or insertions with respect to SEQ ID NO:2.

As used herein, “variants” encompass the following: Variants can differ from the naturally occurring p62 polypeptide in amino acid sequence or in ways that do not involve the sequence, or both. Variants in amino acid sequence are produced when one or more amino acids are substituted with a different natural amino acid, an amino acid derivative or non-native amino acid. When a polynucleotide encoding the protein is expressed in a cell, one naturally occurring amino acid will generally be substituted for another.

Additional sequences that are either at least 85% or about 90% identical over their entire length to polypeptides having the sequences set forth in SEQ ID NO:1 or SEQ ID NO:2 are also provided. The polypeptides that are at least 85% identical to SEQ ID NO:1 can have up to about 59 amino acid additions, deletions, substitutions or insertions and up to about 28 amino acid additions, deletions, substitutions or insertions for SEQ ID NO:2. In the case where a polypeptide is 90% identical to SEQ ID NO:1 the polypeptide may have up to about 39 amino acid additions, deletions, substitutions or insertions and up to about 19 for SEQ ID NO:2.

Methods of Producing Polypeptides of the Invention

Polypeptides of the present invention may be produced using any of several methods well known in the art. For example, the polypeptides may be synthesized using the classic Merrifield solid phase synthesis techniques involving a solid phase method employing Boc-amino acid (Chem. Soc., 85, 2149, (1963)); by using manual or automated procedures, using a solid phase method employing an Fmoc-amino acid (Sheppard, R. C. et al., J. Chem. Soc. Chem. Comm., pp. 165-166 (1985)); using an Advanced Chemtech model 200 available from Advanced Chemtech., Louisville, Ky., using a Millipore 9050+ available from Millipore, Bedford Mass., or other available instrumentation.

Compounds may also be prepared by standard recombinant DNA technology using techniques that are well known in the art. For example, the procedures outlined in Sambrook et al., Molecular Cloning: A Laboratory Manual, 2nd edition, (Cold Spring Harbor Press, Cold Spring Harbor, N.Y.) or Ausubel et al., Current Protocols in Molecular Biology, John Wiley and Sons, New York (1995), both of which are incorporated herein by reference. In other words, polypeptides of the present invention may be produced by incorporating cDNA molecules encoding polypeptides of the invention into functional viral or circular plasmid DNA vectors. These vectors or plasmids can be used to transfect or transform selected microorganisms. The transformed or transfected microorganisms can be cultured under conditions that are conducive to express vector-borne DNA sequences and isolation of the desired polypeptides from the growth medium can be achieved. (See, for example U.S. Pat. No. 5,955,422 incorporated herein by reference.)

After cleavage and deprotienation, compounds of the present invention are purified. For example, gel filtration chromatography and reverse-phase column/HPLC system can be used to purify full length compounds from fragments thereof. The amino acid sequences of the polypeptides produced may be confirmed and identified using standard amino acid analysis, as well as manual and automated Edman degradation and determination of each amino acid. High Pressure Liquid Chromatography (HPLC) analysis and mass spectrometry may also be used to verify the compounds produced.

Computer modeling may be used to design “variants” of fragments p62(1-392) and p62(336-522) based on their preferred structural and functional properties. Polypeptide sequences are analyzed for predicted secondary structure, hydrophobic moment, and amphipathicity. Some computer programs available include Eisenberg Algorithm (Eisenberg et al. Biopolymers 27: 171-177, 1996) for helical structure; Genetics Computer Group (Madison, Wis.) for secondary structure, hydrophobic moment and amphipathizing and Eisenberg et al., Proc. Natl. Sci. USA 4 ed., 81:140-144 (1984) for hydrophobic moment.

Additional polypeptides of the invention are produced by substitutions, additions, deletions or inserts are performed on the parent polypeptide. Conservative substitutions typically include the substitution of one amino acid for another with similar characteristics such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

Other conservative substitutions of amino acids in the p62(1-392) and p62(336-522) fragments can be taken from Table A to produce sequences that are at least 80%, 85% or 90% identical over their entire length to either the p62(1-392) or the p62(336-522) polypeptide. Additional possible substitutions are described by Dayhoff in the Atlas of Protein Sequence and Structure (1988).

TABLE A
Conservative Amino Acid Replacements

| For Amino Acid | Code | Replace with any of |
|---|---|---|
| Alanine | A | D-Ala, Gly, beta-Ala, L-Cys, D-Cys |
| Arginine | R | D-Arg, Lys, homo-Arg, D-homo-Arg, Met, D- |
| | | Met, Ile, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Beta-Ala, Acp |
| Isoleucine | I | D-Ile, Val, D-Val, Leu, D-Leu, Met, D-Met |
| Leucine | L | D-Leu, Val, D-Val, Met, D-Met |
| Methionine | M | D-Met, S-Me-Cys, Ile, D-Ile, Leu, D-Leu, Val, |
| | | D-Val, Norleu |
| Proline | P | D-Pro, L-I-thioazolidine-4-carboxylic acid, D- |
| | | or L-1-oxazoliding-4-carboxylic acid |
| Serine | S | D-Ser, Thr, D-Thr, allo-Thr, Met, D-Met, Met |
| | | (O), D-Met (O), Val, D-Val |
| Tyrosine | Y | D-Try, Phe, D-Phe, L-Dopa, His, D-His |
| Valine | V | D-Val, Leu, D-Leu, Ile, D-Ile, Met, D-Met |

Other variants within the invention are those with modifications which increase polypeptide stability. For example, variants that include residues other than naturally occurring L-amino acids, such as D-amino acids or non-naturally occurring or synthetic amino acids such as beta or gamma amino acids and cyclic variants. Incorporation of D- instead of L-amino acids into the polypeptide may increase its resistance to proteases. See, e.g., U.S. Pat. No. 5,219,990 which is incorporated herein by reference.

Variants with amino acid substitutions which are less conservative may also result in desired derivatives that are at least 80%, 85%, 90% identical over the entire length of the p62(1-392) and the p62(336-522) polypeptides, e.g., by causing changes in charge, conformation and other biological properties. Such substitutions would include for example, substitution of hydrophilic residue for a hydrophobic residue, substitution of a cysteine or proline for another residue, substitution of residue having a small side chain for a residue having a bulky side chain or substitution of a residue having a net negative charge. When the result of a given substitution cannot be predicted with certainty, the derivatives may be readily assayed according to the methods disclosed herein to determine the presence or absence of the desired characteristics.

Just as it is possible to replace substituents of the scaffold, it is also possible to substitute functional groups which decorate the scaffold with groups characterized by similar features. These substitutions will initially be conservative, i.e., the replacement group will have approximately the same size shape, hydrophobicity and charge as the original group. Non-sequence modification may include, for example, in vivo or in vitro chemical derivatization of portions of the protein of this invention, as well as changes in acetylation, methylation, phosphorylation, carboxylation or glycosylation.

In a further embodiment, the polypeptides of the present invention may be modified by chemical modifications in which activity is preserved. For example, the polypeptides may be amidated, sulfated, singly or multiply halogenated, alkylated, carboxylated, or phosphorylated. The polypeptides may also be singly or multiply acylated, such as with an acetyl group, with a farnesyl moiety, or with a fatty acid, which may be saturated, monounsaturated or polyunsaturated. The fatty acid may also be singly or multiply fluorinated. The invention also includes methionine analogs of the polypeptides, for example the methionine sulfone and methionine sulfoxide analogs. The invention also includes salts of the polypeptides, such as ammonium salts, including alkyl or aryl ammonium salts, sulfate, hydrogen sulfate, phosphate, hydrogen phosphate, dihydrogen phosphate, thiosulfate, carbonate, bicarbonate, benzoate, sulfonate, thiosulfonate, mesylate, ethyl sulfonate and benzensulfonate salts.

Production of Vectors and Expression Thereof

In order to express a biologically active p62(1-392) or p62(336-522) or variants thereof, the nucleotide sequences encoding these polypeptides may be inserted into appropriate expression vector, i.e., a vector which contains the necessary elements for the transcription and translation of the inserted coding sequence.

Methods which are well known to those skilled in the art may be used to construct expression vectors containing sequences encoding polypeptides of the invention and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, En Plainview, N.Y., chs. 4, 8, and 16-17; and Ausubel, F. M. et al. (1995, and periodic supplements) Current Protocols in Molecular Biology, John Wiley & Sons, New York, N.Y., chs. 9, 13, 16.)

A variety of expression vector/host systems may be utilized to contain and express sequences encoding the polypeptides of the invention. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The invention is not limited by the host cell employed.

The vectors may include a “control element” or “regulatory sequence.” A “control element” or “regulatory sequence” are those non-translated regions, e.g., enhancers, promoters, and 5′ and 3′ untranslated regions, of the vector and polynucleotide sequences encoding the polypeptides of the invention which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters, e.g., hybrid lacZ promoter of the BLUESCRIPT phagemid (Stratagene, La Jolla Calif.) or PSPORT1 plasmid (GIBCO/BRL), may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding polynucleotides of the invention, vectors based on SV40 or EBV may be used with an appropriate selectable marker.

In bacterial systems, a number of expression vectors may be selected depending upon the use intended for the polypeptides of the invention. For example, when large quantities of these polypeptides are needed to quickly enhance the activation of NF-6B or to quickly increase the translocation of NF-6B vectors which direct high level expression of fusion proteins that are readily purified may be used. Such vectors include, but are not limited to, multifunctional E. coli cloning and expression vectors such as BLUESCRIPT (Stratagene), in which the sequence encoding NHLP may be ligated into the vector in frame with sequences for the amino-terminal Met and the subsequent 7 residues of .beta.-galactosidase so that a hybrid protein is produced, and pIN vectors. (See, e.g., Van Heeke, G. and S. M. Schuster (1989), J. Biol. Chem. 264: 5503-5509.) pGEX vectors (Pharmacia Biotech, Uppsala, Sweden) may also be used to express foreign polypeptides as fusion proteins with glutathione S-transferase (GST). In general, such fusion proteins are soluble and can easily be purified from lysed cells by absorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. (See, e.g., Ausubel, supra; and Grant et al. (1907) Methods Enzymol. 153: 516-544, which is herein incorporated by reference.)

In cases where plant expression vectors are used, the expression of sequences encoding polypeptides of the invention may be driven by any of a number of promoters. For example, viral promoters such as the 35S and 19S promoters of CaMV may be used alone or in combination with the omega leader sequence from TMV. (Takamatsu, N. (1097) EMBO J. 6: 307-311). Alternatively, as stated above, plant promoters such as the small subunit of RUBISCO or heat shock promoters may be used. (See. e.g., Coruzzi, G. et al. (1984) EMBO J. 3: 1671-1680; Broglie, R. et al. (1984) Science 224: 838-843; and Winter, J. et al. (1991) Results Probl. Cell Differ. 17: 85-105) These constructs can be introduced into plant cells by direct DNA transformation or pathogen-mediated transfection. Such techniques are described in a number of generally available reviews. (See, e.g., Hobbs, S. or Murry, L. E. in McGraw Hill Yearbook of Science and Technology (1992) McGraw Hill, new York, N.Y.; pp. 191-196, which is herein incorporated by reference.)

As stated above, an insect system may also be used to express polypeptides of the invention. For example, in one such system, A. californica nuclear polyhedrosis virus (AcNPV) may be used as a vector to express foreign genes in S. fugiperda cells or in T. larvae. The sequences encoding the polypeptides of the invention may be cloned into a non-essential region of the virus, such as the polyhedrin gene, and placed under control of the polyhedrin promoter. Successful insertion of sequences encoding the polypeptides will render the polyhedrin gene inactive and produce recombinant virus lacking coat protein. The recombinant viruses may then be used to infect, for example, S. frugiperda cells or T. larvae in which [p. 62(1-392), p. 62(336-522) or other polypeptides of the invention may be expressed. (See, e.g., Engelhard, E. K. et al. (1994) Proc. na. Acad. Sci. 91: 3224-3227, which is herein incorporated by reference.)

As stated above, in mammalian host cells a number of viral-based expression systems may be utilized. In cases where an adenovirus is used as an expression vector, sequences encoding NHLP may be ligated into an adenovirus transcription/translation complex consisting of the late promoter and tripartite leader sequence. Insertion in a non-essential E1 or E3 region of the viral genome may be used to obtain a viable virus which is capable of expressing anyone of the polypeptides of the invention in infected host cells. (See, e.g., Logan, J. and T. Shenk (1984) Proc. Natl,. Acad. Sci. 81: 3655-3659, which is herein incorporated by reference.) In addition, transcription enhancers, such as the rous sarcoma virus (RSV) enhancer, may be used to increase expression in mammalian host cells.

Human artificial chromosomes (Has) may also be employed to deliver larger fragments of DNA than can be contained and expressed in a plasmid. Has of about 6 kb to 10 Mb are constructed and delivered via conventional delivery methods (lipsomes, polycationic amino polymers, or vesicles) for therapeutic purposes.

Specific initiation signals may also be used to achieve more efficient translation of sequences encoding p62(1-392), p62(336-522) or variants thereof. Such signals include the ATG initiation codon and adjacent sequences. In cases where sequences encoding the polypeptides and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be natural or synthetic. The efficiency of expression may be enhanced by the inclusion of enhancers appropriate for the particular cell system sued. (See, e.g., Scharf, D. et al. (1994) Results Probl. Cell Differ. 20: 125-162.)

In addition, a host cell strain may be chosen for its ability to control expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, 93T and WI38), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of expressing the polypeptides of the invention can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to convert resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes and adenine phosphoribosyltransferase genes, which can be employed in tk.sup.-or apr.sup.-cells, respectively. (Se, e.g., wigler, M. et al. (1997) Cell 11: 223-232; and Lowy, I. et al. (1980) Cell 22: 817-823). Also, antimetabolite, antibiotic, or herbicide resistance can be used as the basis for selection. For example, dhfr confers resistance to methotrexate; npt confers resistance to the aminoglycosides neomycin and G-418; and als or pat confer resistance to chlorsulfuron and phosphinotricin acetyltransferase, respectively. (See, e.g., Wigler, M. et al. (1980) Proc. Natl. Acad. Sci. 77: 3567-3570; Colbere-Garapin, F. et al (1981) J. Mol. Biol. 150: 1-14; and Murry, supra.) Additional selectable genes have been described, e.g., trpB, which allows cells to utilize indole in place of tryptophan, or hisD, which allows cells to utilize hisinol in place of histidine. (See, e.g., Hartman, S. C. and R. C. Mulligan (1988) Proc. Natl. Acad. Sci. 85: 8047-8051.) Visible markers, e.g., anthocyanins, beta glucuronidase and its substrate GUS, luciferase and its substrate luciferin may be used. Green fluorescent proteins (GFP) (Clontech, Palo Alto, Calif.) can also be used. These markers can be used not only to identify transformants, but also to quantify the amount of transient or stable protein expression attributable to a specific vector system. (See, e.g., Rhodes, Calif. et al. (1995) methods Mol. Biol. 55: 121-131.)

Although the presence/absence of marker gene expression suggests that the gene of interest is also present, the presence and expression of the gene may need to be confirmed. For example, if the polynucleotide sequence encoding p62(1-392) is inserted within a marker gene sequence, transformed cells containing the polynucleotide sequences encoding p62(1-392) can be identified by the absence of marker gene function. Alternatively, a marker gene can be placed in tandem with a sequence encoding p62(1-392) under the control of a single promoter. Expression of the marker gene in response to induction or selection usually indicates expression of the tandem gene as well.

Alternatively, host cells which contain the nucleic acid sequence encoding p62(1-392) and express p62(1-392) may be identified by a variety of procedures known to those of skill in the art. These procedures include, but are not limited to, DNA-DNA or DNA-RNA hybridization and protein bioassay or immunoassay techniques which include membrane, solution, or chip based technologies for the detection and/or quantification of nucleic acid or protein sequences.

The presence of polynucleotide sequences encoding the polypeptides of the invention can be detected by DNA-DNA or DNA-RNA hybridization or amplification using probes or fragments of polynucleotide sequences encoding the polypeptides. Nucleic acid amplification based assays involve the use of oligonucleotides or oligomers based on the polynucleotide sequences encoding the polypeptides of the invention, to detect transformants containing DNA or RNA encoding these polypeptides.

A variety of protocols for detecting and measuring the expression of polypeptides of the invention, using either polyclonal or monoclonal antibodies specific for the protein, are known in the art. Examples of such techniques include enzyme-linked immunosorbent assays (ELISAs), radioimmunoassays (RIAs), and flurorescence activated cell sorting (FACs). A two-site, monoclonal-based immunoassay utilizing monoclonal antibodies reactive to two non-interfering eptiopes on p62(1-392), p62(336-522) or variants thereof is preferred, but a competitive binding assay may be employed. These and other assays are well described in the art. (See, e.g., Hampton, R. et al. (1990) Serological Methods, a Laboratory manual, ASP Press, St. Paul, Minn., Section IV; and Madden, D E et al. (1983) J. Exp. Med. 158: 1211-1216, which are herein incorporated by reference).

A wide variety of labels and conjugation techniques are known by those skilled in the art and may be used in various nucleic acid and amino acid assays. Means for producing labeled hybridization or PCR probes for detecting sequences related to polynucleotides encoding polypeptides of the present invention include oligolabeling, nick translation, end-labeling, or PCR amplification using a labeled nucleotide. Alternatively, the sequences encoding these polypeptides, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding polypeptides of the invention may be cultured under conditions suitable for the expression and recovery of the polypeptide from cell culture. The polypeptide produced by a transformed cell may be secreted or contained intracellularly depending on the sequence and/or the vector used. As will be understood by those of skill in the art, expression vectors containing polynucleotides which encode the polypeptides of the invention may be designed to contain signal sequences which direct secretion of these polypeptides through a prokaryotic or eukaryotic cell membrane. Other constructions may be used to join sequences encoding the polypeptides of the invention to nucleotide sequences encoding a polypeptide domain which will facilitate purification of soluble proteins.

Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunix Corp., Seattle, Wash.). The inclusion of cleavable linker sequences, such as those specific for Factor XA or enterokinase (Invitrogen, San Diego, Calif.), between the purification domain and the polypeptide encoding sequence may be used to facilitate purification. One such expression vector provides for expression of a fusion protein containing polypeptides of the invention and a nucleic acid encoding 6 histidine residues preceding a thioredoxin or an enterkinase cleavage site. The histidine residues facilitate purification on immobilized metal ion affinity chromatography (IMAC). (Se, e.g., Porath, J. et al. (1992) Prot. Exp. Purif. 3: 263-281.) The enterokinase cleavage site provides a means for purifying NHLP from the fusion protein. (See, e.g., Kroll, D. J. et al. (1993) DNA Cell Biol. 12: 441-453.)

Pharmaceutical Compositions/Salts

The polypeptides of the present invention and/or salts thereof may also be formulated with pharmaceutically acceptable carriers to provide pharmaceutical compositions. Such a pharmaceutical composition may contain an enhancing NF-6B activating effective amount of the p62(1-392) polypeptide or one or more of the p62(1-392) polypeptide variants or pharmaceutical acceptable salts thereof. An enhancing NF-6B activating effective amount is an amount of the p62(1-392) polypeptide or one or more of the p62(1-392) polypeptide variants that is sufficient to enhance the amount of activated NF-6B in an individual host cell as compared to the amount of NF-6B that is activated in a cell that is not subjected to any amount of the p62(1-392) or one or more of the p62(1-392) polypeptide variants. The amount of NF-6B activated in a cell can be measured by measuring the amount of free I6B in the cell. To measure the amount of Free I6B, the I6B can be tagged with a marker that is detectable once it dissociates from the NF-6B molecule.

Other pharmaceutical compositions may contain a NF-6B translocation inhibiting amount of the p62(366-522) polypeptide or one or more of the p62(366-522) polypeptide variants of the polypeptides of the present invention or pharmaceutical acceptable salts thereof. An NF-6B translocation inhibiting effective amount is an amount of the p62(366-522) polypeptide or one or more of the p62(366-522) variants that is sufficient to inhibit the translocation of NF-6B across a nuclear membrane in an individual host cell as compared to the amount of NF-6B translocated across a nuclear membrane of a cell that is not subjected to any amount of the p62(366-522) polypeptide or one or more of the p62(366-522) polypeptide variants. The amount of NF-6B translocated across a nuclear membrane of a cell can be observed by tagging the NF-6B with a marker and detecting the marker.

Pharmaceutically acceptable acid addition salts of the compositions comprising one or more of the polypeptides of the present invention may also be used to make the pharmaceutical compositions. These include salts derived from nontoxic inorganic acids such as hydrochloric, nitric, phosphoric, sulfuric, hydrobromic, hydriodic, hydrofluoric, phosphorous, and the like. Other pharmaceutically acceptable acid addition salts of the present invention include for example salts derived from nontoxic organic acids, such as aliphatic mono- and dicarboxylic acids, phenyl-substituted alkanoic acids, hydroxyl alkanoic acids, alkanedioic acids, aromatic acids, aliphatic and aromatic sulfonic acids, etc. Such salts thus include sulfate, pyrosulfate, bisulfate, sulfite, bisulfate, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, phenylacetate, citrate, lactate, tartrate, methanesulfonate, and the like. Also contemplated are salts of amino acids such as arginate and the like and gluconate, galacturonate and the like (see, for example, Berge, S. M., et al., Pharmaceutical Salts, Journal of Pharmaceutical Science, 66:1-19, 1977, which are herein incorporated by reference).

Also, the basic nitrogen-containing groups of the present polypeptides in the pharmaceutical compositions can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained. All of these forms are within the scope of the present invention.

Pharmaceutically acceptable base addition salts may also be used in the pharmaceutical compositions. These base addition salts are formed with metals or amines, such as alkali and alkaline earth metals or organic amines. Examples of metals used as cations are sodium, potassium, magnesium, calcium, and the like. Examples of suitable amines are N,N(-dibenzylethylenediamine, chloroprocaine, choline, diethanolamine, dicyclohexylamine, ethylenediamine, N-methylglucamine, and procaine.

The base addition salts of these acidic compounds are prepared by contacting the free acid form with a sufficient amount of the desired base to produce the salt in the conventional manner. Certain compounds of the present invention can exist in unsolvated forms as well as solvated forms, including hydrated forms. In general, the solvated forms, including hydrated forms, are equivalent to unsolvated forms and are intended to be encompassed within the scope of the present invention.

The carriers used in the pharmaceutical compositions of the invention are the non-toxic, inert pharmaceutically suitable carriers normally used, for example, solid, semi-solid or liquid diluents, fillers and formulation auxiliaries of all types. Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In a preferred embodiment, the pharmaceutical compositions of the invention are formulated in unit dosage forms. The unit dosage forms of the present invention include for example tablets, dragees, capsules, caplets, pills, granules and suppositories. Tablets, dragees, capsules, caplets, pills and granules can contain the active compounds of the invention in addition to the customary excipients, such as (a) fillers and extenders, for example, starches, lactose, sucrose, glucose, mannitol and silicic acid, (b) binders, for example, carboxymethylcellulose, alginates, gelatin and polyvinylpyrrolidone, (c) humectants, for example, glycerol, (d) disintegrating agents, for example, agar-agar, calcium carbonate and sodium carbonate, (e) solution retarders, for example, paraffin; (f) absorption accelerators, for example, quaternary ammonium compounds; (g) wetting agents, for example, acetyl alcohol and glycerol monostearate; (h) absorbents, for example, kaolin and bentonite; and (i) lubricants, for example, talc, calcium stearate, magnesium stearate and solid polyethylene glycols, or mixtures of the substances listed under (a) to (i) directly hereinabove.

The tablets, capsules, caplets, pills and granules can be provided with the customary coatings and shells, optionally containing pacifying agents and can also be of such composition that they release the active compounds only or preferentially in a certain part of the intestinal tract, optionally in a delayed manner. Examples of embedding compositions which can be used are polymeric substances and waxes.

The active polypeptides of the invention may be present in microencapsulated form, if appropriate with one or more of the above mentioned excipients. The active polypeptides may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any nontoxic, physiologically acceptable and metabolizable lipid capable of forming liposomes can be used. The present compositions in liposome form can contain, in addition to a compound of the invention, stabilizers, preservatives, excipients, and the like. The preferred lipids are phospholipids and phosphatidyl cholines (lecithins), both natural and synthetic. Methods to form liposomes are known in the art.

Suppositories for rectal administration of the polypeptides of the invention can be prepared by mixing the compounds with a suitable nonirritating excipient such as cocoa butter and polyethylene glycols which are solid at ordinary temperatures but liquid at the rectal temperature and will therefore melt in the rectum and release the compound. Suppositories can contain, in addition to the active polypeptides, the customary water-soluble or water-insoluble excipients, for example, polyethylene glycols, fats, for example, cacao fat and higher esters (for example, C(14)-alcohol with C(16)-fatty acid), or mixtures of these substances.

The polypeptides of the invention can also be formulated as ointments, pastes, creams and gels. These topical mixtures can contain, in addition to the active polypeptides, the customary carriers, for example, animal and vegetable fats, waxes, paraffins, starch tragacanth, cellulose derivatives, polyethylene glycols, silicones, bentonites, silicic acid, talc and zinc oxide, or mixtures of these substances.

The polypeptides of the invention may also be formulated as dusting powders and sprays. As with the topical mixtures alone, the dusting powders and sprays can contain, in addition to the active compounds, the customary carriers, for example, lactose, talc silicic acid, aluminum hydroxide, calcium silicate and polyamide powder, or mixtures of these substances. Sprays can additionally contain customary propellants, for example, chlorofluorohydrocarbons.

Topical compositions may be contained on transdermal patches or iontophoresis devices. In particular, the topical compositions may include one or more of the compounds of the present invention in a pharmaceutically acceptable vehicle containing up to about 70% w/w of the active agent, preferably up to about 50% w/w of the active agent. Particular embodiments of the invention include injection solutions, solutions and suspensions for oral therapy, gels, pour-on formulations, emulsions, drops, ophthalmological and dermatological formulations, silver salts and other salts, ear drops, eye ointments, powders or solutions that can be used for local therapy.

Liquid dosage forms for oral administration may include pharmaceutically acceptable emulsions, solutions, suspensions, syrups, and elixirs containing inert diluents commonly used in the art, such as water. Such compositions may also comprise adjutants, such as wetting agents, emulsifying and suspending agents, and sweetening, flavoring, and perfuming agents. Solutions and emulsions can contain, in addition to the active compounds, customary excipients, such as solvents, solubilizing agents and emulsifiers, for example, water, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, glycerol formal, tetrahydrofurfuryl alcohol, polyethylene glycols and fatty acid esters of sorbitan, or mixtures of these substances.

For parenteral administration, the solutions and emulsions can also be in a sterile form which is isotonic with blood. Suspensions can contain, in addition to the active compounds, customary excipients, such as liquid diluents, for example, water, ethyl alcohol or propylene glycol and suspending agents, for example ethoxylated isostearyl alcohols, polyoxyethylene sorbitol and sorbitan esters, microcrystalline cellulose, aluminum methydroxide, bentonite, agar-agar, and tragacanth, or mixtures of these substances. Injectable solutions contain about 500 mg to about 5000 mg of active ingredients per liter of solution. Preferably, the injectable solution contains about 1000 mg to about 3000 mg of the active ingredients per liter of solution. Most preferably, the solution contains about 500 mg to about 1000 mg of the active ingredient per liter of solution.

The above mentioned pharmaceutical compositions may also contain other pharmaceutically active compounds in addition to the claimed polypeptides of the invention. The aforementioned pharmaceutical formulations are prepared in the customary manner by known methods, for example, by mixing the active compound or compounds with the carrier or carriers.

Administration of the Compositions

The pharmaceutical compositions of the present invention can be administered parenterally, enterally or topically. In addition, the compositions can also be administered transdermally. It will be obvious to those skilled in the art that the following dosage forms may comprise as the active component, one or more peptides of the present invention or a corresponding pharmaceutically acceptable salt of a peptide of the present invention.

Pharmaceutical compositions containing the p62(1-392) polypeptide or variants thereof may be administered to a mammal to increase the amount of activated NF-6B in the mammal. This method of administration comprises, administering to the mammal a pharmaceutical composition comprising, in combination with a pharmaceutical acceptable carrier, a NF-6B activating effective amount of the peptide having the formula I or variants thereof. As stated above, a NF-6B activating effective amount is an amount of the pharmaceutical composition containing at least one polypeptide of the invention that increases the amount of activated NF-6B in a cell that is not subjected to any amount of the polypeptide. This amount is easily quantified by measuring the amount of free NLS in the mixture.

In addition, pharmaceutical compositions containing the p62(336-522) polypeptide, or variants thereof, may be administered to a mammal to inhibit the translocation of NF-6B across a membrane in the mammal. This method of administration comprises, administering to the mammal a pharmaceutical composition comprising, in combination with a pharmaceutical acceptable carrier, a NF-6B translocation inhibiting effective amount of the peptide having the formula III or variants thereof. As stated above, a NF-6B translocation inhibiting effective amount is an amount of a pharmaceutical composition that contains at least one polypeptide of the invention that inhibits translocation of activated NF-6B across a nuclear membrane as compared to the amount of activated NF-6B that is translocated across the membrance that is not subjected to the pharmaceutical composition.

In addition, an effective amount refers to that amount of the active ingredient, e.g. a p62(1-392) or p62(336-522) polypeptide of the invention, which ameliorates the symptoms or condition. Therapeutic efficacy and toxicity may be determined by standard pharmaceutical procedures in cell cultures or with experimental animals, such as calculating the ED50(the does therapeutically effective in 50% of the population) or the LD50 (the dose lethal in 50% of the population) statistics. The dose ratio of therapeutic to toxic effects is the therapeutic index, and it can be expressed as the ED50/LD50 ratio. Pharmaceutical compositions which exhibit large therapeutic indices are preferred. The data obtained from cell culture assays and animal studies are used to formulate a range of dosage forms for human use. The dosage contained in such compositions is preferably within a range of circulating concentrations that includes the ED50 with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or bi-weekly depending on the half-life and clearance rate of the particular formulation.

Generally, treatment is initiated using the standard regiment of starting with small dosages substantially less than the optimum dose of the pharmaceutical composition containing one or more of the peptides of the present invention or pharmaceutical acceptable salts thereof. Thereafter, the dosage is increased by small increments until the optimum effect under the circumstances is reached.

Normal dosage amounts may vary from about 10 mg to 100 mg, up to a total dose of about 500 mg, depending upon the route of administration. Guidance as to particular dosages and methods of delivery is provided in the literature and generally available to practitioners in the art. Those skilled in the art will employ different formulations for polynucleotides than for polyproteins or their inhibitors. Similarly, delivery of polynucleotides or polypeptides will be specific to particular cells, conditions, locations. etc.

The present invention may be administered to a mammal, such as a rat, cat, dog, monkey, mouse, mammal and more particularly a human.

The pharmaceutical compositions of the invention can be administered as the sole active pharmaceutical agent, or they can also be administered in combination with one or more molecules. Thus, although the formulations disclosed herein above are effective and relatively safe medications either inhibiting NF-6B translocation or activating NF-6B without inhibiting translocation, the possible concurrent administration of these formulations with other medications or agents to obtain beneficial results is not excluded. Such other agents may be the agents associated with gene therapy.

When administered as a combination, the therapeutic agents can be formulated as separate compositions that are given at the same time or different times, or the therapeutic agents can be given as a single composition. Collectively, the most important determinant of potency of compositions based on the polypeptide sequences of the present invention are the novel nature of the amino acid groups themselves. Within the sequences, the stereochemistry yields active compounds, but it is possible that compounds containing minor additions to either/both N- and C-terminal to the polypeptide sequences group may improve the bioavailability, solubility, resistance to animal, plant or bacterial proteases, metabolic stability or other pharmacodynamic effects.

For administration by injection, it is preferred to use the polypeptides of the invention or salts thereof dissolved in a sterile aqueous vehicle which may also contain other solutes such as buffers or preservatives as well as sufficient quantities of pharmaceutically acceptable salts or of glucose to make the solution isotonic. Injectable preparations include, for example, sterile injectable aqueous or oleaginous which suspensions may be formulated according to the known art using suitable dispersing or wetting and suspending agents. The sterile injectable preparation may also be a sterile injectable solution or suspension in a nontoxic parenterally acceptable diluent or solvent, such as for example 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In another embodiment, the present invention provides novel microlaboratory systems and methods that are useful for performing high-throughput screening assays. In particular, the present invention provides methods of using such devices that are useful in screening large number of different compounds for their effects on a variety of chemical, and preferably, biochemical systems.

In order to provide methods and devices for screening compounds for effects on biochemical systems, the present invention generally incorporates model in vitro systems which mimic a given biochemical system in vitro for which effector compounds are desired. The range of systems against which compounds can be screened and for which effector compounds are desired, is extensive. For example, compounds may be screened for effects in blocking, slowing or otherwise inhibiting events associated with biochemical systems. For example, test compounds may be screened for their ability to inhibit the translocation of activated NF-6B across the nuclear membrane of a cell which in turn inhibits the binding of the NF-6B to the transcriptional control region of a particular gene. Thus, shortening off the gene. Alternatively, molecules that cause the dissociation of I6B from NF-6B molecule may also be detected. Compounds which show promising results in these screening assay methods can then be subjected to further testing to identify effective pharmacological agents for the treatment of disease or systems of a disease.

However, in some instances, high-throughput screening assays are not able to detect weak signals of interaction between the test compounds and the biochemical system being tested unless the signals are above a specific baseline level. For example, a weak activation of NF-6B may not be detected by a detecting system unless it is above a particular baseline level because the detecting means is not sensitive enough to detect such low levels of activity. In particular, a compound capable of enhancing the activation of NF-6B may not be detected in an assay if the level of activation is below a certain baseline level that is specific to the detecting means. Alternatively, the level of translocation of activated NF-6B across a nuclear membrane may also be too low to detect.

To overcome these problems, the assay of the present invention is provided with at least one of the polypeptides selected from the group consisting of a polypeptide having the amino acid sequence set forth in SEQ ID NO:1, a polypeptide having the amino acid sequence that is at least 80% identical over its entire length to a polypeptide set forth in SEQ ID NO:1, a polypeptide having the amino acid sequence set forth in SEQ ID NO:2, and a polypeptide having the amino acid sequence that is at least 80% identical over its entire length to a polypeptide set forth in SEQ ID NO:2.

One or all of these polypeptides enhance the particular activity, i.e., translocation of NF-6B across a nuclear membrane or activation of NF-6B, above a baseline level so that minor charges in activity caused by the compound being tested can be detected by the detecting system used in the high throughput screening assay. For example, a discrete amount of polypeptide of the invention listed above may activate NF-6B above a baseline level below which the detecting mechanism of the assay is unable to detect effects by test compounds, so that activation of additional NF-6B molecules by the test compounds is now detectable by the detecting system.

Test compounds that have only a minor effect on the activation of NF-6B, e.g., the liberation of I6B from the NF-6B exposing the NLS on the NF-6B, might not be detected by a highthrough put assay that does not contain the polypeptides of the invention because the amount of I6B released from NF-6B may be below the sensitivity level of the detecting system. The assay of the present invention uses the polypeptides of the invention to “prime” the detecting system to be more sensitive to minor changes the amount of free I6B directly indicating the amount of NF-6B activation caused by the test compound. In other words, the detecting system detects the effect of the polypeptides of the invention on NF-6B activation as a background level, and any additional effect caused by the test compound is now within the sensitivity range of the detecting system.

So too is true with effects of test compounds on the activated NF-6B translocation across a nuclear membrane. Test compounds having only a minor effect on NF-6B translocation.

In one embodiment the screening assay is used to screen for effectors of a ligand/receptor interaction. An assay used for screening for effectors of a receptor/ligand interaction include incubating a receptor/ligand binding pair in the presence of a test compound. The level of binding of the receptor/ligand pair is then compared to negative and/or positive controls where a decrease in normal binding is seen, the test compound is determined to be an inhibitor of the receptor/ligand binding where an increase in that binding is seen, the test compound is determined to be an enhancer or inducer of the interaction.

The assay may be conducted in standard 96 well assay plate in a single reaction vessel or custom plate may be manufactured using a number of microfabrication techniques that are well known in the art. These techniques include injection molding or stamp molding methods where large numbers of substrates may be produced using e.g., rolling stamps to produce large sheets of microscale substrates or polymer microcasting techniques where the substrate is polymerized within a micromachined mold.

The examples below are provided to illustrate the subject invention and are not included for the purpose of limiting the invention.

Experimental Details

a. Plasmids and Constructs

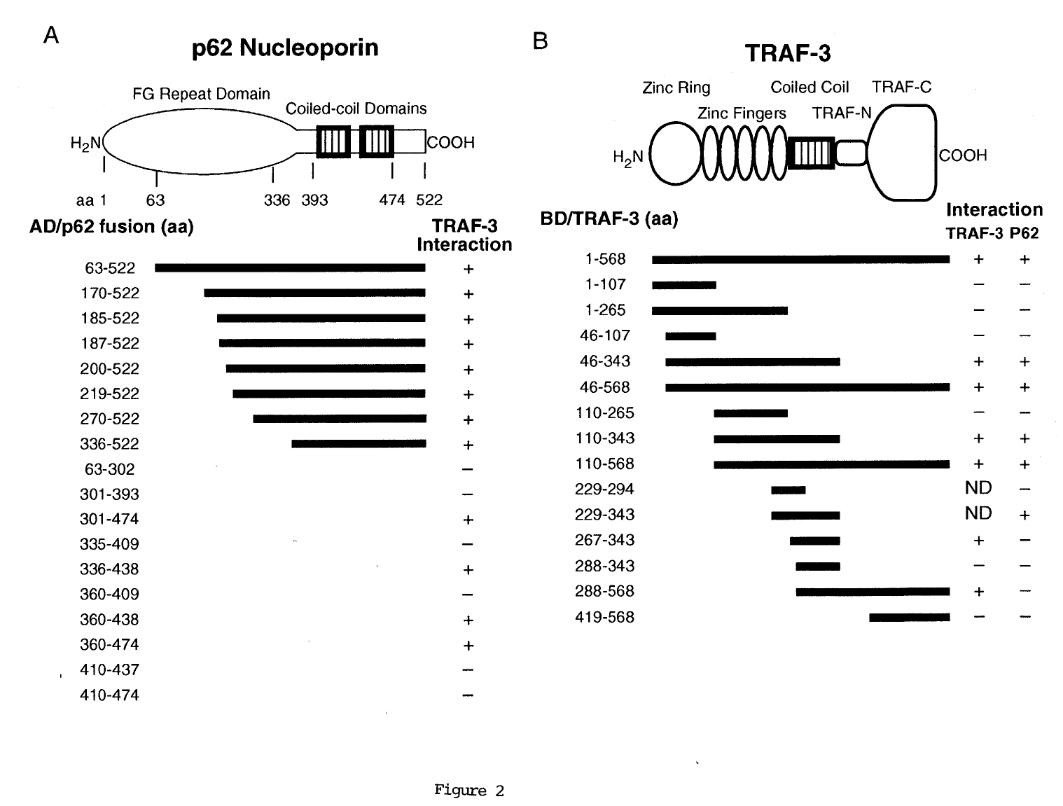

The full open reading frame (ORF) of human TRAF-3 or selected sub-domains were amplified by PCR using oligonucleotide primers flanked by unique restriction sites using Expand HF polymerase mix (Roche Molecular Biochemicals, Indianapolis, Ind.) and cloned into the pCR2.1 TA cloning vector (Invitrogen, Carlsbad, Calif.). All PCR products were sequenced and found to match the expected products based on published sequences. Full-length TRAF-3 was also subcloned into pGBT9 and pGAD424 expression vectors from the Matchmaker Two-Hybrid system (Clontech Laboratories) to generate a GAL4 DNA-binding domain (BD)/TRAF-3 fusion construct (pGBT9/TRAF-3) and a GALA Activation domain (AD)/TRAF-3 fusion construct (pGAD424/TRAF-3). TRAF-3 subdomains were subcloned into pGBT9 generating the constructs shown in FIG. 2B.

TRAF-3 was also subcloned into pEBVHisB (Invitrogen) to allow mammalian expression with an amino-terminal poly-histidine (His)-tag and X-press epitope, and into pCEP4 (Invitrogen) for expression of non-tagged TRAF-3. The control construct pEBVhis LacZ, encoding His/X-press tagged β-galactosidase (β-gal) was obtained from Invitrogen. Full-length cDNAs encoding TRAF-1, TRAF-4, TRAF-5 and TRAF-6 were each amplified by PCR, cloned into pCR2.1 and subsequently subcloned into pGBT9 and pEBVHis (e.g., pEBVHis/TRAF-2). A restriction fragment encoding p62 nucleoporin amino acids (aa) 336-522 obtained in the yeast two-hybrid screen was subcloned into pBluescriptII SK (Stratagene, La Jolla, Calif.), then subcloned into pCGN to allow mammalian expression with an N-terminal hemagglutinin (HA) tag (pCGN/p62(336-522)). The full ORF of 1-gal was amplified from pEBVHis LacZ by PCR and cloned into pCGN (pCGN/LacZ) for use as a control.

Full-length p62 was amplified by RT-PCR from Jurkat T-cell mRNA and TA cloned into pCR2.1. Deletion mutants of p62 were generated by subcloning of restriction fragments of p62 obtained in several yeast two-hybrid clones into the pGAD424 activation domain vector (Clontech Laboratories). An oligonucleotide linker encoding an HA tag and stop codon was ligated into pCR2.1/p62 at a unique SacI site to generate a truncated p62(aa 1-392) construct with a C-terminal HA tag. Full-length p62, HA-tagged p62(1-392), and HA-tagged p62(336-522) were subcloned into pCEP4 for mammalian expression. PRDIIx4 Luc was generated by subcloning the NF-6B sites and interferon beta promoter from PRDIIx4 CAT into the pGL3 Enhance plasmid (Promega, Madison, Wis.). PRLtk (Promega) which constitutively expresses Renilla Luciferase was used to control for transfection efficiency in reporter assays. DNA for mammalian transfection was prepared with Maxiprep and Megaprep columns (Qiagen, Valencia, Calif.).

b. Yeast Two-Hybrid Screening for TRAF-3 Interacting Proteins

An EBV transformed B-cell cDNA library in pACT (Clontech Laboratories, Palo Alto, Calif.) that was used for yeast two-hybrid screening. Yeast phenotype was measured by colony lift β-gal filter assay (blue is β-gal*) and growth on Synthetic Dropout (SD) media lacking histidine (His) and containing 50 mM 3-AT to inhibit leaky expression of the HIS3 reporter gene product. Interaction of BD- and AD-fusion proteins in the Matchmaker Two-Hybrid system (yeast strain Y190) results in expression of β-gal and HIS3 Reporter genes yielding a β-gal* His* yeast phenotype. Transfected Y190 yeast colonies that grew in the absence of His and expressed β-gal were recovered and expanded. Plasmid DNA was isolated from these yeast colonies and pACT plasmids were recovered by transfection of E. coli HB101 and growth on M9 minimal media containing ampicillin (Sigma Chemical Co., St. Louis, Mo.) but lacking leucine. Recovered plasmids were retransfected into yeast with either empty pGBT9 or pGBT9/TRAF-3 to confirm capacity to induce reporter gene expression.

c. Characterization of p62/TRAF Protein Interactions

The pGBT9/TRAF-1 through 6 constructs were transfected with empty pGAD424 or with pACT/p62(336-522) into Y190. Yeast transfected with pGBT9/TRAF-1 alone or with either activation domain vector displayed constitutive activation of the β-gal and His reporters in the two-hybrid system, suggesting that this TRAF1 fusion construct may harbor a cryptic transactivation domain, and this TRAF1 construct was not used in any further analyses. Colonies from the other TRAF constructs were tested for β-gal activity by colony lift assay. Prior to scoring colonies for growth in the absence of His, colonies were streaked onto plates lacking His and containing 50 mM 3-AT and grown for 3 days. Colonies from this plate were then patched in duplicate onto plates either containing or lacking His but containing 50 mM 3-AT and grown for an additional 3 days before scoring for growth.

d. Tissue Culture and Transient Transfection for Protein Expression

293T cells were grown in Iscove's Modified Dulbecco's Media (IMDM) (Mediatech, Herndon, Va.) supplemented with 10% Fetal Bovine Serum (FBS) (Mediatech), and 100× penicillin-streptomycin (Sigma Chemical Co.) (10% IMDM). Mammalian expression vectors described above were transfected into 293T cells using the calcium phosphate technique, Mammalian Cell Transfection Kit (Specialty Media, Lavallette, N.J.). 293T cells were seeded at 10⁶ cells/100 mm per dish 1 day prior to transfection in 10 ml of 10% IMDM. Media was replaced with fresh media prior to transfection. Precipitates were generated using 10 μg of each expression construct and applied to the cells. After 12 hours of culture at 37° C., the media was replaced with fresh media and cells were cultured for an additional 24 to 36 hours.

e. Protein Analysis

Transfected 293T cells in 100 mm dishes were washed with 1× phosphate buffered saline (PBS) and lysed by addition of μl of ice cold NP-40 Lysis Buffer containing; 1% Nonidet P40 (Fluka Chemie, Buchs, Switzerland, 50 mm Tris pH 7.4 (Sigma Chemical Co.), 150 mM sodium chloride (Fisher Scientific Pittsburgh, Pa.), 40 mM sodium fluoride (Fisher Scientific), 100 μM sodium orthovanadate (Sigma Chemical Co.), 1 μg/ml Aprotinin (Sigma Chemical Co.), 1 μg/ml Leupeptin (Sigma Chemical Co.), 1 μg/ml Pepstatin (Sigma Chemical Co.), and 0.5 mM PMSF (Sigma Chemical Co.). Cells were scraped from the plate and transferred to microcentrifuge tubes. Samples were incubated on ice for 30 minutes, before centrifugation at 16,000 g for 15 minutes at 4° C. Cleared lysates were transferred to new tubes and quantitated using the Detergent Compatible Protein Assay (Biorad Laboratories, Hercules, Calif.). Equal amounts of protein, typically 400 μg, were precleared with washed Protein-G Sepharose (Pharmacia Biotech, Piscataway, N.J.) for 1 hour at 4° C.

Cleared supernatants were than incubated with 2 μg of anti-HA (3C10) (Roche Molecular Biochemicals) or 3 μl of anti-X-press (Invitrogen) and rocked at 4° C. for 1 hour. 20 μl of washed Protein G beads were then added and the samples were rocked for 1 hour. Beads were washed 3 times with NP-40 Lysis Buffer, then 3 times with 1×PBS. Supernatants were aspirated and the beads were boiled in 40 μl of 1×SDS PAGE buffer for 5 minutes. Samples were separated by 10% SDS PAGE and transferred to Immobilon-P PVDF membranes (Millipore Corporation, Bedford, Mass.). Membranes were washed with 1× Tris Buffered Saline (TBS) containing 50 mM Tris pH 7.5 (Fisher Scientific) and 150 mM sodium chloride (Fisher Scientific), and then blocked in 1% Blocking Reagent (Roche Molecular Biochemicals) dissolved in 1×TBS.

Membranes were probed with anti-Xpress, diluted 1:5000 in 0.5% Blocking Reagent, or anti-HA (12CA5) at 0.4 μg/ml (Roche Molecular Biochemicals) in 0.5% Blocking Reagent. Membranes were incubated for 1 hour, then washed twice with TBS containing 0.1% Tween-20 (Sigma Chemical Co.) (1× TBST) and twice with 0.5% Blocking Reagent. Membranes were incubated with anti-mouse IgG-Peroxidase (Roche Molecular Biochemicals) at 1:2500 in 0.5% Blocking Reagent for 60 minutes at room temperature, then washed 4 times in 1× TBST. Proteins were detected with the BM Chemiluminescence Blotting Substrate (POD) (Roche Molecular Biochemicals). Following detection, membranes were stripped of antibodies by incubation with stripping solution containing 62.5 mM Tris pH 6.7, 2% SDS, and 50 mM 2-mercaptoethanol (Fisher Scientific) for 30 minutes at 50° C. The membranes were rinsed with distilled water and then with 1×TBS. The stripped membranes were blocked and reprobed with antibodies as described above.

f. Luciferase Reporter Assays

293T cells were seeded at 5×10⁵ cells per well in 6 well dishes containing 5 ml 10% IMDM. Cells were transfected by the calcium phosphate method as described above, except each culture was transfected with 3 μg of the indicated expression vector, 300 ng of PRDIIx4 Luc, and 75 ng of pRLtk. Where indicated, cells were co-transfected with either 400 ng of pCEP/CD40 or 100 ng of pCDNA3/p65(RelA). Total DNA content of each sample was normalized with empty pCEP4 expression vector. Precipitates were applied and cells were cultured 15 hours at 37° C. before media was replaced. Cells were washed with 1×PBS 36 hours after transfection before lysis in 1× Passive Lysis Buffer and assayed using the Dual Luciferase Assay Kit (Promega, Madison, Wis.). Firefly luciferase reporter levels were normalized for transfection efficiency with the internal Renilla luciferase control. Values were scaled to the level of Firefly luciferase activity observed in parallel cultures transfected with empty pCEP4. Data shown are representative of 3 independent experiments and error bars represent the standard deviation of measurements from triplicate cultures.

Results

a. Yeast Two-Hybrid Screen for TRAF-3 Interacting Molecules

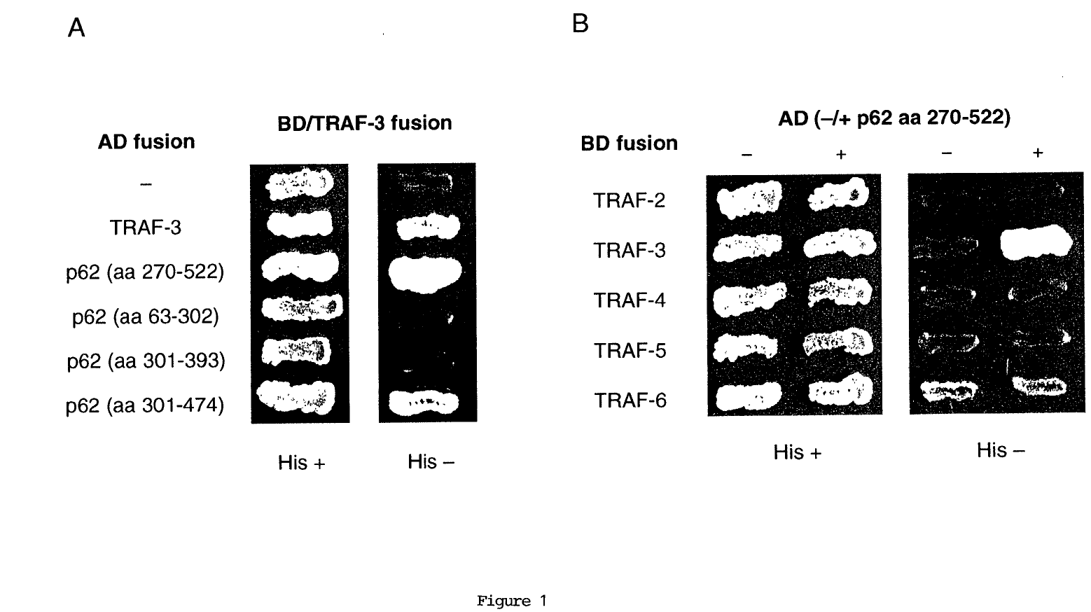

To identify molecules that interact TRAF-3, a yeast two-hybrid screen was performed using a “bait” construct comprising the full-length TRAF-3 ORF fused to the GAL4 DNA binding domain (BD) (PGBT9/TRAF-3). This construct was determined to be appropriate for screening since transfection of pGBT9/TRAF-3 in the yeast two-hybrid system, either alone or with control GALA activation domain (AD) fusion proteins, failed to induce either 1-gal expression or growth on His⁻ selective media. To ensure that the TRAF-3 fusion protein was functionally expressed, pGBT9/TRAF-3 was co-transfected with an AD/full-length TRAF-3 fusion construct (pGAD424/TRAF-3) and resulted in both induction of β-gal activity and growth on His⁻ media, consistent with TRAF-3 homo-oligomerization (FIG. 1A). Therefore, the pGBT9/TRAF-3 construct was used to screen a cDNA library from EBV transformed B-cell line. This screen yielded 51 clones that reproducibly required the presence of the BD/TRAF-3 construct to induce β-gal activity and support growth on His⁻ media. Restriction digest mapping and sequencing of selected clones followed by dot blot hybridization of all clones with gene specific probes revealed that 34 clones encode portions of p62 Nucleoporin (FIG. 1A). The 34 clones encoding p62 represent at least 10 independently generated cDNAs based on the presence of distinct 5′-termini in the p62 ORF (summarized in FIG. 2A).

To determine whether p62 interacts with other TRAF family members, a clone encoding p62 as 270-622 (pACT/p62(270-522)) was tested for interaction with other TRF family members using the yeast two-hybrid system. In these studies, p62(270-522) interacted with t3 but not TRAF-2, -4, -5, or -6 (FIG. 1B). The specificity of p62 binding for TRAF-3 is notable, given the relatively high level of co-liner homology that TRAF-3 shares with TRAF-5. The inability of p62(270-522) to interact with TRAF-5 (pGBT9/TRAF-5) was not due to lack of 3 expression, because co-transfection of pGBT9/TRAF-5 and pGAD424/TRAF-3 resulted in induction of β-gal activity and growth on His⁻ media, consistent with the TRAF-3:TRAF-5 interaction that has been demonstrated by other biochemical methods (Pullen et al., 1998). Therefore, these data indicate that the interaction of p62 with TRAF-3 is specific and restricted to TRAF-3.

Although the N-termini of the p62 fusion proteins obtained in the yeast-hybrid screen range from p62 aa 64 to 336, all TRAF-3 interacting p62 clones contain the p62 C-terminus, suggesting that the two predicted C-terminal coiled-coil domains of p62 (aa 372-406 and 430-457) (Wolf et al., 1997) are responsible for interacting with TRAF-3. To more precisely map the p62 domain that interacts with TRAF-3, deletion mutants of p62 lacking portions of the C-terminus were generated and tested for interaction with TRAF-3 in the yeast two-hybrid system (FIG. 1A). Constructs encoding p62 aa 270-522 and aa 302-474 are able to interact with TRAF-3, however p62(302-391) lacks the ability to bind TRAF-3. These data indicate that the C-terminal 48 aa of p62 are dispensable for TRAF-3 binding, and suggest that the p62 coiled-coil domains mediate TRAF-3 binding.

To map the regions of TRAF-3 required for p62 interaction, deletion mutants of TRAF-3 were generated and tested for interaction with p62 in the yeast two-hybrid system. As controls, the TRAF-3 fragments were tested in parallel for interaction with full-length TRAF-3, since the coiled-coil domain and TRAF-C domains are known to play roles in TRAF-3 oligomerization. The smallest fragment of TRAF-3 tested which retains the ability to interact with p62 encodes the Zn fingers and coiled-coil domain encompassing aa 110-343 (FIG. 2B). Smaller fragments of TRAF-3 encoding only the Zn fingers (aa 110265) or coiled-coil domain (aa 267-343) alone were unable to interact with p62(336-522). The lack of interaction of TRAF-3(267-343) with p62 was not due to lack of expression, since TRAF-3(267-343) was able to associate with full-length TRAF-3. Together these data show that the TRAF-3 Zn finger and coiled-coil domains are both necessary and sufficient for p62 binding.

b. Interactions of TRAF-3 and p62 in Mammalian Cells

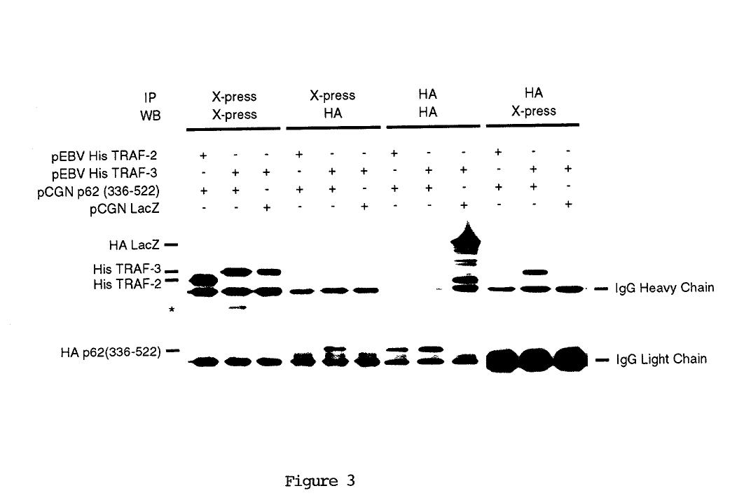

To determine whether TRAF-3 and p62 interact in mammalian cells, His-tagged TRAF-3 (pEBVHis/TRAF-3) and HA-tagged p62(336-522) (pCGN/p62(355-522)) were over-expressed in 293T cells. As controls for the specificity of TRAF-3 binding to p62, His-tagged TRAF-2 (pEBVHis/TRAF-2) or HA-tagged P-gal (pCGN/LacZ) were co-transfected with pCGN/p62(336-522) or pEBVHis/TRAF-3, respectively, in parallel cultures. Immunoprecipitation of His-TRAF-3 resulted in the co-immunoprecipitation of RA-p62(336-522) (FIG. 3). The His-TRAF-3 association with HA-p62(336-522) is specific since His-TRAF-3 fails-to co-immunoprecipitate RA-LacZ and His-TRAF-2 fails to immunoprecipitate RA-p62(336-522). Reciprocally, immunoprecipitation of ILk-p62(336-522) co-immunoprecipitates His-TRAF-3. The RA-p62(336-522) association with His-TRAF-3 is specific, since immunoprecipitation of RA-p62(336-522) fails to immunoprecipitate His-TRAF2 and HA-LacZ fails to co-immunoprecipitate His-TRAF-3. Together, these data indicate that TRAF-3 and p62 are able to specifically associate in mammalian cells.

c. Effects of p62 Fragments on RelA Translocation

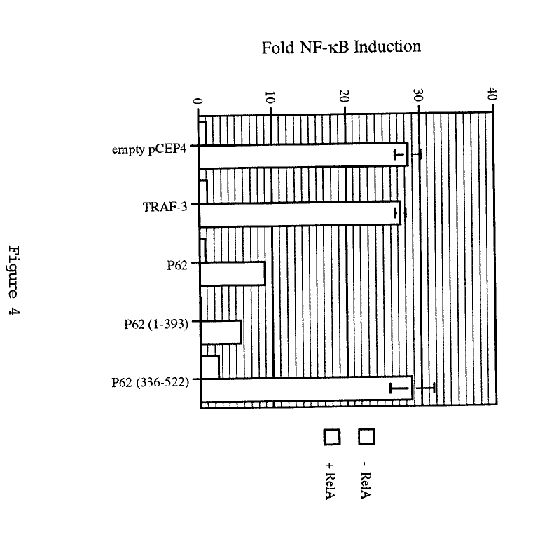

To study the functional effects of p62 on NF-6B activation and translocation, the first series of experiments evaluated the effects of over-expressing p62 fragments on the translocation of activated NF-6B. Over-expression of RelA results in excess free RelA which is able to dimerize, translocate to the nucleus, and activate transcription of NF-6B responsive genes in the absence of other stimuli (FIG. 4). Since the N-terminal domain of p62 is known to bind the translocation factor p10/NFT2 (Clarkson et al., 1996) and the C-terminal domain binds karyopherin-β (Percipalle et al., 1997) as well as TRAF-3, the effects of over-expressing an N-terminal fragment p62(1-391) were compared with those of a C-terminal fragment p62(336-522), as well as full-length p62 on RelA-induced NF-6B reporter activity. Over-expression of p62(1-392) inhibits RelA-induced NF-6B reporter activity, consistent with an effect of p62(1-392) on translocation of activated NF-6B. In contrast, over-expression of p62(336-522) did not alter RelA-induced NF-6B reporter activity. The effect of over-expressing full length p62 was intermediate between p62(1-392) and p62(336-522). Cells transfected with full-length p62 and p62(1-392) showed similar morphology and viability to samples transfected with other constructs. In addition, Western blot analysis of lysates used in luciferase assays showed similar levels of RelA expression in p62-, p62(1-392)-, and p62(336-522)-transfected cells (data not shown). Together, these data indicate that p62(1-392), and to a lesser extent full-length p62, inhibit nuclear translocation of activated NF-6B, consistent with a depletion of nuclear translocation factors that bind the N-terminus of p62, In addition, these data indicate that the TRAF-3 binding fragment, p62(336-522), does not measurably alter the translocation of activated NF-6B and suggest that this fragment may be used to study effects of p62(336-522) on NF-6B activation.

d. Effects of p62 Fragments on Inducing NF-6B Activation

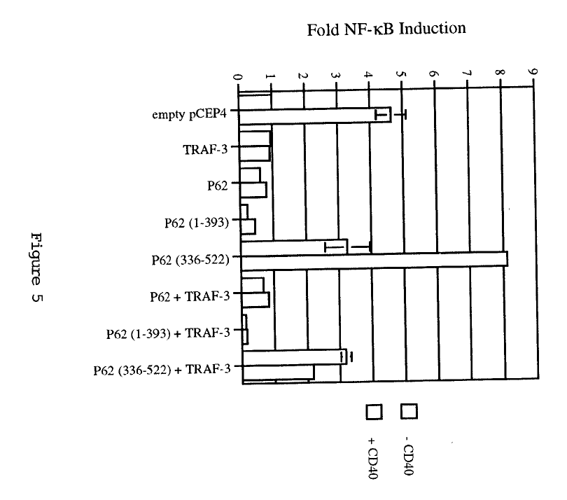

To study whether the TRAF-3 binding fragment, p62(336-522) regulates NF-6B activation, the functional effects of over-expressing p62(336-522) on baseline and CD40-induced NF-6B activation were studied in 293T cells. In parallel experiments, p62(1-392) and full-length p62 were also studied, although it was expected that their effects on inhibiting the translocation of activated NF-6B would inhibit the NF-6B reporter assay. In fact, p62(1-392) and to a lesser extent, full-length p62, inhibit baseline and CD40-induced reporter activity, consistent with their inhibition of NF-6B translocation (FIG. 5). In contrast, over-expression of the TRAF-3 binding fragment, P62(336-522) induces approximately 3-fold activation of NF-6B reporter gene activity in the absence of CD40 expression (FIG. 5) and approximately 8-fold activation of reporter gene expression when co-transfected with CD40, which is approximately twice the level observed in cells transfected with CD40 and empty pCEP4 expression vector (FIG. 5). Together with the previous finding that p62(336-522) does not affect the translocation of activated NF-6B (FIG. 4), these data suggest that p62(336-522), which contains the TRAF-3 binding domain, induces NF-6B activation.

These studies also evaluated the effects of over-expressing full-length TRAF-3 on p62(336-522)-induced NF-6B activation. Consistent with previous reports, over-expression of full-length TRAF-3 has little effect on background NF-6B reporter gene activity but inhibits the NF-6B activation induced by over-expression of CD40 (FIG. 5). Full-length TRAF-3 fails to inhibit p62(336-522)-induced NF-6B activation (FIG. 5). In addition, co-expression of full-length TRAF-3 with p62(336-522) and CD40 results in NF-6B reporter activity that is approximately equal to the level induced by p62(336-522) alone (FIG. 5). This finding is consistent with the interpretation that full-length TRAF-3 inhibits the component of NF-6B activation induced by CD40 over-expression, but not the component induced by p62(336-522) expression. Co-expression of full-length TRAF-3 with p62(1-392) or full-length p62 resulted in reporter gene activity approximately equivalent to samples expressing either of these p62 constructs alone. Together, these findings suggest that the p62(336-522) effects on inducing NF-6B activation are downstream of the inhibitory effects that full-length TRAF-3 exerts on CD40-triggering.

Discussion

These studies on the mechanisms by which TRAF-3 mediates signal transduction show that TRAF-3 interacts with p62 nucleoporin and that p62 fragments have distinct effects on NF-6B activation and translocation. TRAF-3's ability to bind p62 is not shared by TRAF-2, TRAF-4, TRAF-5, or TRAF-6. The p62 C-terminal coiled-coiled domains were found to mediate interactions with TRAF-3. The p62 C-terminal domain is known also to act as a docking site for import complexes by binding karyopherin-β (Rexach and Blobel, 1995; Percipalle et al., 1997) and the N-terminal domain is known to interact with p10/NTF2, an essential factor of the nuclear import machinery (Paschal and Gerace, 1995; Clarkson et al., 1996). To separate these effects, functional studies were performed on N-terminal and C-terminal fragments of p62. Over-expression of the p62 N-terminal fragment inhibits NF-6B activation in a dominant negative manner, consistent with inhibition of nuclear translocation. In contrast, the p62 C-terminal TRAF-3 binding domain induces NF-6B activation which suggests a previously unappreciated role for p62 in NF-6B activation.

The role of p62 in NF-6B translocation has been inferred from the fact that cytoplasmic NF-6B complexes must translocate to the nucleus in order to activate transcription of target genes and that p62 is known to serve as a docking site for karyopherin-complexed transcription factors. The exposed NLS of activated NF-6B binds karyopherin-α and this complex binds E karyopherin-β, which docks the NF-6B/karyopherin-α/β complex to p62 at the nuclear pore (Nadler et al., 1997; Torgerson et al., 1998). In resting cells, NF-6B is rendered inactive by interactions with I-κB which masks the NF-6B NLS. Signaling by cell surface receptors leads to the dissociation of I-κB and the liberation of NF-6B with an exposed NLS (Beg et al., 1992). Over-expression of the NF-6B protein RelA bypasses I-κB regulation because free RelA is expressed in excess of cellular Iκ-B.

Consequently, over-expressed RelA undergoes nuclear translocation and activates transcription in the absence of exogenous stimuli that normally dissociate I-κB from the pool of inactive NF-6B. Therefore, the reporter gene activity induced by over-expression of RelA is a measure of p62 mediated nuclear translocation that does not depend on dissociation of I-κB. The finding that over-expression of an N-terminal p62 domain, p62(1-392), inhibits RelA-induced NF-6B reporter gene activity suggests a dominant negative effect by p62(1-392) on p62-mediated translocation of RelA. Since the N-terminus of p62 binds p10/NTF2 in vitro (Clarkson et al., 1996) and NTF2 is essential for nuclear import (Paschal and Gerace, 1995), it is believed that over-expression of p62(1-392) in the cytoplasm depletes cellular pools of p10/NTF2, to inhibit translocation of RelA import complexes through the nuclear pore. Thus, these data are consistent with the known requirement for nuclear translocation of RelA in order to exert effects on target gene transcription.

[contrast to the inhibitory effect of the p62 N-terminal fragment on RelA translocation, over-expression of a C-terminal fragment fails to inhibit RelA translocation. The lack of inhibition by p62(336-522) is surprising because this region is known to be sufficient to bind karyopherin-β (Percipalle et al., 1997) and thus might have been expected to inhibit docking of n karyopherin/RelA complexes to endogenous p62 at the nuclear pore, particularly since over expression of the p62 C-terminal domain has been reported to lead to significant accumulation outside the nuclear pore complex (Starr et al., 1990; Carmo-Fonseca et al., 1991). It is unclear why over-expression of p62(336-522) fails to inhibit RelA translocation, but may relate to pore-associated p62 having a higher avidity than cytoplasmic p62(1-392) for karyopherin-β. However, the finding that p62(356-522) does not inhibit nuclear translocation of RelA provided an opportunity to stud y its effects on NF-6B activation.

[-expression of p62(336-522) induces NF-6B reporter gene activity in the absence of CD40 signaling and augments the effect of CD40 over-expression on NF-6B reporter gene activity. Since over-expression of a p62 C-terminal fusion protein is known to result in expression of substantial amounts of the p62 fusion protein outside the nuclear pore complex in the cytoplasm (Carmo-Fonseca et al., 1991), the ability of p62(336-522) to increase NF-6B dependent reporter gene activity arises from triggering the cytoplasmic signaling cascade that liberates activated NF-6B from I-κB. The effects of full-length p62 over-expression were intermediate between those of the individual N-terminal and C-terminal fragments both on translocation of RelA and on activating NF-6B. The intermediate effects of full-length p62 are consistent with mixed effects, in which the p62 C-terminal domain induces NF-6B activation and the N-terminal domain inhibits nuclear translocation.

[finding that the C-terminal p62 fragment induces NF-6B activation was unexpected. Since the p62 C-terminal fragment binds TRAF-3, it is interesting to consider how TRAF-3 binding may relate to its effect. Consistent with previous work, full-length TRAF-3 over-expression is known to inhibit NF-6B activation triggered by CD40 over-expression (Rothe et al., 1995). Since over-expression of full-length TRAF-3 fails to affect translocation of over-expressed RelA (see FIG. 4), full-length TRAF-3 appears to act proximal to nuclear translocation in the receptor-triggered signaling cascade. In addition, full-length TRAF-3 fails to inhibit p62(336-522)-induced NF-6B activation, which suggests that the inhibitory effect of full-length TRAF-3 on CD40-induced NF-6B activation is also proximal to the inducing effect of p62(336-522). Therefore, over-expression of full-length TRAF-3 may inhibit CD40-induced NF-6B activation at the level of TRAF-3 binding to the CD40 cytoplasmic tail, the recruitment of other factors to CD40-bound TRAF-3, or by the formation of homo-trimers of full-length TRAF-3.

[has recently been shown that over-expression of certain TRAF-3 splice-deletion variants induces NF-6B activation (van Eyndhoven et al., 1999). In contrast to the effects of over-expressing full-length TRAF-3 alone, co-expression of full-length TRAF-3 with the activating splice-deletion variants augments NF-6B activation induced by the splice deletion isoforms alone, suggesting that full-length TRAF-3 stabilizes mixed TRAF-3 trimeric complexes that consist of full-length and splice-deletion TRAF-3 isoforms (van Eyndhoven et al., 1999). Together these observations suggest that TRAF-3 heterotrimers are capable of forming signaling complexes that induce NF-6B activation. Therefore, one possible explanation for the NF-6B inducing activity of p62(336-522) is that it interacts with such TRAF-3 heterotrimers and activates them. In this regard, TRAF-3 is also known to interact with NIK that plays a role in activating NF-6B (24;25). The finding that over-expression of full-length TRAF-3 fails to inhibit p62(336-522) effects, suggests that p62 interactions with TRAF-3 trimers are transient and that each p62 molecule may activate multiple TRAF-3 complexes.

[considerations suggest biological roles for TRAF-3:p62 interactions in signaling. By binding TRAF-3, p62 may recruit TRAF-3 signaling complexes to the nuclear pore. Such recruitment would result in local activation of NF-6B signaling complexes. In this regard, the NF-6B signalosome consisting of IKK and associated molecules has been shown to contain RelA (Mercurio et al., 1997; Heilker et al., 1999). Therefore, the local activation of the signalosome (e.g. by TRAF-3 bound NIK) at the nuclear pore complex may liberate activated NF-6B in close proximity to the karyopherin-β docking site on p62, perhaps facilitating its import. In addition, recruitment of a TRAF-3 signaling complex containing kinases (such as TRAF-3 bound ASK1 (Nishitoh et al., 1998)) to the nuclear pore might result in phosphorylation of p62. Such modification of p62 has been observed (Macaulay et al., 1995; Buss et al., 1994) and correlates with nuclear import of a different transcription factor (Buss et al., 1994). Understanding the roles of TRAF-3:p62 binding and the potential activation of TRAF-3 and p62 are important goals of future research.

[data may also relate to the essential function that TRAF-3 signaling is known to play in T-dependent antibody production. These observations suggest that the essential roles of TRAF-3 in signaling may be due to its unique ability among TRAF family members to associate with p62. In addition to p62's known roles in mediating nuclear translocation of NF-6B, the present study suggests that p62:TRAF-3 interactions may be a means by which p62 organizes a signaling complex at the nuclear pore and in which p62 induces NF-6B activation. In this regard, certain clinically important anti-inflammatory and immunosuppressive agents, such as acetylsalicylic acid and cyclosporin A, are believed to function by inhibiting steps required for nuclear translocation of the transcription factors NF-6B (Yin et al., 1998) and NF-AT (Emmel et al., 1989), respectively. These considerations indicate that TRAF-3:p62 interactions provide a novel target for therapeutic agents that may regulate immune responses.

[the invention has been illustrated and described with respect to specific illustrative embodiments and modes of practice, it will be apparent to those skilled in the art that various modifications and improvements may be made without departing from the scope and spirit of the invention. Accordingly, the invention is not to be limited by the illustrative embodiments and modes of practice.

### REFERENCES

Arch, R. H. and Thompson, C. B. (1998). 4-1BB and Ox40 are members of a tumor necrosis factor (TNF)—nerve growth factor receptor subfamily that bind TNF receptor-associated factors and activate nuclear factor κB. Mol. Cell Biol. 18, 558-565.
Attar, R. M., Macdonald-Bravo, H., Raventos-Suarez, C., Durham, S. K., and Bravo, R. (1998). Expression of constitutively active I-κB P in T cells of transgenic mice: persistent NF-6B activity is required for T-cell immune responses. Mol. Cell Biol. 18, 477-487.
Baeuerle, P. A. and Baltimore, D. (1996). NF-6B: ten years after. Cell 87, 13-20.
Becraft, P. W., Stinard, P. S., and McCarty, D. R. (1996). CRINKLY4: A TNFR-like receptor kinase involved in maize epidermal differentiation. Science 273, 1406-1409.
Beg, A. A., Ruben, S. M., Scheinman, R. I., Haskill, S., Rosen, C. A., and Baldwin, A. S. J. (1992). I-κB interacts with the nuclear localization sequences of the subunits of NF-6B: a mechanism for cytoplasmic retention. Genes Dev. 6, 1899-1913.
Berberich, I., Shu, G. L., and Clark, E. A. (1994). Cross-linking CD40 on B cells rapidly activates nuclear factor-xB. J. Immunol. 153, 4357-4366.
Boucher, L. M., Marengere, L. E., Lu, Y., Thukral, S., and Mak, T. W. (1997). Binding sites of cytoplasmic effectors TRAF1, 2, and 3 on CD30 and other members of the TNF receptor superfamily. Biochem. Biophys. Res. Commun. 233, 592-600.
Bus, F., Kent, H., Stewart, M., Bailer, S. M., and Hanover, J. A. (1994). Role of different domains in the self-association of rat nucleoporin p62. J Cell Sci. 107, 631-638.
Carmo-Fonseca, M., Kern, H., and Hurt, E. C. (1991). Human nucleoporin p62 and the essential yeast nuclear pore protein NSP1 show sequence homology and a similar domain organization. Eur J. Cell Biol. 55, 17-30.
Cheng, G. and Baltimore, D. (1996). TANK, a co-inducer with TRAF2 of TNF- and CD40L-mediated NF-6B activation. Genes Dev. 10, 963-973.
Cheng, G., Cleary, A. M., Ye, Z-s., Hong, D. I., Lederman, S., and Baltimore, D. Involvement of CRAF1, a relative of TRAF, in CD40 signaling. Science 267, 1494-1497. 1995.
Clarkson, W. D., Kent, H. M., and Stewart, M. (1996). Separate binding sites on nuclear transport factor 2 (NTF2) for GDP-Ran and the phenylalanine-rich repeat regions of nucleoporins p62 and Nsplp. J. Mol. Biol. 263, 517-524.
Dadgostar, H. and Cheng, G. (1998). An intact zinc ring finger is required for tumor necrosis factor receptor-associated factor-mediated nuclear factor-xB activation but is dispensable for c-Jun N-terminal kinase signaling. J. Biol. Chem. 273, 24775-24780.
DiDonato, J. A., Hayakawa, M., Rothwarf, D. M., Zandi, E., and Karin, M. (1997). A cytokine-responsive I-xB kinase that activates the transcription factor NF-6B. Nature 388, 548-554.
Emmel, E. A., Verweij, C. L., Durand, D. B., Higgins, K. M., Lacy, E., and Crabtree, G. R. (1989). Cyclosporin A specifically inhibits function of nuclear proteins involved in T cell activation. Science 246, 1617-1620.
Finlay, D. R., Meier, E., Bradley, P., Horecka, J., and Forbes, D. J. (1991). A complex of nuclear pore proteins required for pore function. J. Cell Biol. 114, 169-183.
Foxce, W. R., Cheung, T. C., and Ware, C. F. (1997). Dominant negative mutants of TRAF3 reveal an important role for the coiled coil domains in cell death signaling by the lymphotoxin-β receptor. J. Biol. Chem. 272, 30835-30840.
Gedrich, R. W., Gilfillan, M. C., Duckett, C. S., Van Dongen, J. L., and Thompson, C. B. (1996). CD30 contains two binding sites with different specificities for members of the tumor necrosis factor receptor-associated factor family of signal transducing proteins. J. Biol. Chem. 271, 12852-12856.
Gorlich, D., Vogel, F., Mills, A. D., Hartmann, E., and Laskey, R. A. (1995). Distinct functions for the two importin subunits in nuclear protein import. Nature 377, 246-248.
Grumont, R. J., Rourke, I. J., O'Reilly, L. A., Strasser, A., Miyake, K., Sha, W., and Gerondakis, S. (1998). B lymphocytes differentially use the Rel and nuclear factor kappaB1 (NF-6B) transcription factors to regulate cell cycle progression and apoptosis in quiescent and mitogen-activated cells. J. Exp. Med 187, 663-674.
Heilker, R., Freuler, F., Pulfer, R., Di Padova, F., and Eder, J. (1999). All three I-κB isoforms and most Rel family members are stably associated with the I-κB kinase 1/2 complex. Eur J. Biochem. 259, 253-261.
Horwitz, B. H., Zelazowski, P., Shen, Y., Wolcott, K. M., Scott, M. L., Baltimore, D., and Snapper, C. M. (1999). The p65 subunit of NF-6B is redundant with p50 during B cell proliferative responses, and is required for germline CH transcription and class switching to IgG3. J. Immunol. 162, 1941-1946.
Hostager, B. S., Hsing, Y., Harms, D E, and Bishop, G. A. (1996). Different CD40-mediated signaling events require distinct CD40 structural features. J. Immunol. 157, 1047-1053.
Hsing, Y. and Bishop, G. A. (1999). Requirement for nuclear factor-κB activation by a distinct subset of CD40-mediated effector functions in B lymphocytes. J. Immunol. 162, 2804-2811.
Hu. H. M., O'Rourke, K., Boguski, M. S., and Dixit, V. M. (1994). A novel RING finger protein interacts with the cytoplasmic domain of CD40. J. Biol. Chem. 269, 30069-30072.
Kawamata, S., Hori, T., Imura, A., Takaori-Kondo, A., and Uchiyama, T. (1998). Activation of 0X40 signal transduction pathways leads to tumor necrosis factor receptor-associated factor (TRAF) 2- and TRAF5-mediated NF-6B activation. J. Biol. Chem. 273, 5808-5814.
Lederman, S., Cleary, A. M., Yellin, M. J., Frank, D. M., Karpusas, M., Thomas, D. W., and Chess, L. (1996). The central role of the CD40-ligand and CD40 pathway in T-lymphocyte-mediated differentiation of B lymphocytes. Curr Opin. Hematol. 3, 77-86.
Macaulay, C., Meier, E., and Forbes, D. J. (1995). Differential mitotic phosphorylation of proteins of the nuclear pore complex. J. Biol. Chem. 270, 254-262.
Malinin, N. L., Boldin, M. P., Kovalenko, A. V., and Wallach, D. (1997). MAP3K-related kinase involved in NF-6B induction by TNF, CD95 and IL-1. Nature 385, 540-544.
Mercurio, F., Zhu, H., Murray, B. W., Shevchenko, A., Bennett, B. L., Li, J., Young, D. B., Barbosa, M., Mann, M., Manning, A., and Rao, A. (1997). IKK-1 and IKK-2: cytokine-activated I-κB kinases essential for NF-6B activation. Science 278, 860-866.
Mosialos, G., Birkenbach, M., Yalamanchili, R., VanArsdale, T., Ware, C., and Kieff, E. (1995). The Epstein-Barr virus transforming protein LMP1 engages signaling proteins for the tumor necrosis factor receptor family. Cell 80, 389-399.
Nadler, S. G., Tritschler, D., Haffar, O. K., Blake, J., Bruce, A. G., and Cleaveland, J. S. (1997). Differential expression and sequence-specific interaction of karyopherin-α with nuclear localization sequences. J. Biol. Chem. 272, 4310-4315.
Nishitoh, H., Saitoh, M., Mochida, Y., Takeda, K., Nakano, H., Rothe, M., Miyazono, K., and Ichijo, H. (1998). ASK1 is essential for JNK/SAPK activation by TRAF2. Mol. Cell 2, 389-395.
Park, Y. C., Burkitt, V., Villa, A. R., Tong, L., and Wu, H. (1999). Structural basis for self-association and receptor recognition of human TRAF2. Nature 398, 533-538.
Paschal, B. M. and Gerace, L. (1995). Identification of NTF2, a cytosolic factor for nuclear import that interacts with nuclear pore complex protein p62. J. Cell Biol. 129, 925-937.
Percipalle, P., Clarkson, W. D., Kent, H. M., Rhodes, D., and Stewart, M. (1997). Molecular interactions between the importin-α/β heterodimer and proteins involved in vertebrate nuclear protein import. J. Mol. Biol. 266, 722-732.
Pullen, S. S., Miller, H. G., Everdeen, D. S., Dang, T. T. A., Crute, J. J., and Kehry, M. R. (1998). CD40-tumor necrosis factor receptor-associated factor (TRAF) interactions: regulation of CD40 signaling through multiple TRAF binding sites and TRAF hetero-oligomerization. Biochemistry. 37, 11836-11845.
Regnier, C. H., Song, H. Y., Gao, X., Goeddel, D. V., Cao, Z., and Rothe, M. (1997). Identification and characterization of an I-κB kinase. Cell 90, 373-383.
Rexach, M. and Blobel, G. (1995). Protein import into nuclei: association and dissociation reactions involving transport substrate, transport factors, and nucleoporins. Cell 83, 683-692.
Rothe, M., Sarma, V., Dixit, V. M., and Goeddel, D. V. (1995). TRAF2-mediated activation of NF-6B by TNF Receptor 2 and CD40. Science 269, 1424-1427.
Rothe, M., Xiong, J., Shu, H. B., Williamson, K., Goddard, A., and Goeddel, D. V. (1996). I-TRAF is a novel TRAF-interacting protein that regulates TRAF-mediated signal transduction. Proc. Natl. Acad. Sci. U. S. A. 93, 8241-8246.
Sato, T., Irie, S., and Reed, J. C. (1995). A novel member of the TRAF family of putative signal transducing proteins binds to the cytosolic domain of CD40. FEBS Letters 358, 113-118.
Snapper, C. M., Zelazowski, P., Rosas, F. R., Kehry, M. R., Tian, M., Baltimore, D., and Sha, W. C. (1996). B cells from p50/NF-6B knockout mice have selective defects in proliferation, differentiation, germ-line CH transcription, and Ig class switching. J. Immunol. 156, 183-191.
Song, H. Y., Regnier, C. H., Kirschning, C. J., Goeddel, D. V., and Rothe, M. (1997). Tumor necrosis factor (TNF)-mediated kinase cascades: bifurcation of nuclear factor-κB and c-jun N-terminal kinase (JNK/SAPK) pathways at TNF receptor-associated factor 2. Proc. Natl. Acad. Sci. U.S. A. 94F, 9792-9796.
Starr, C. M., D'Onofrio, M., Park, M. K., and Hanover, J. A. (1990). Primary sequence and heterologous expression of nuclear pore glycoprotein p62. J. Cell Biol. 110, 1861-1871.
Torgerson, T. R., Colosia, A. D., Donahue, J. P., Lin, Y. Z., and Hawiger, J. (1998). Regulation of NF-6B, AP-1, NFAT, and STAT1 nuclear import in T lymphocytes by noninvasive delivery of peptide carrying the nuclear localization sequence of NF-6B p50. J. Immunol. 161, 6084-6092.
Van Eyndhoven, W. G., Gamper, C. J., Cho, E., Mackus, W. J. M., and Lederman, S. (1999). TRAF-3 mRNA splice-deletion variants encode isoforms that induce NF-6B activation. Mol. Imm. in press.
Vanarsdale, T. L., VanArsdale, S. L., Force, W. R., Walter, B. N., Mosialos, G., Kieff. E., Reed, J. C., and Ware, C. F. (1997). Lymphotoxin-β receptor signaling complex: role of tumor necrosis factor receptor-associated factor 3 recruitment in cell death and activation of nuclear factor KB. Proc. Natl. Acad. Sci. U.S.A. 94, 2460-2465.
Wolf, E., Kim, P. S., and Berger, B. (1997). MultiCoil: a program for predicting two- and three-stranded coiled coils. Protein Sci. 6. 1179-1189.
Woronicz, J. D., Gao, X., Cao, Z., Rothe, M., and Goeddel, D. V. (1997). I-κB kinase-β: NF-6B activation and complex formation with I-κB kinase-β and NIK. Science 278, 866-869.
Xu, Y., Cheng, G., and Baltimore, D. (1996). Targeted disruption of TRAF3 leads to postnatal lethality and defective T-dependent immune responses. Immunity 5, 407-415.
Yamamoto, H., Kishimoto, T., and Minamoto, S. (1998). NF-6B activation in CD27 signaling: involvement of TNF receptor-associated factors in its signaling and identification of functional region of CD27. J. Immunol. 161, 4753-4759.
Yin, M. J., Yamamoto, Y., and Gaynor, R. B. (1998). The anti-inflammatory agents aspirin and salicylate inhibit the activity of I-κB kinase-β. Nature 396, 77-80.

### BACKGROUND OF THE INVENTION

Abbreviations: TRAF, TNF receptor-associated factor; NF-κB, Nuclear factor-KB; TNF-R, Tumor Necrosis Factor Receptor; IVT, in vitro transcription; NIK, NF-κB-inducing kinase; ASK1, Apoptosis Signaling-regulated Kinase 1; IκB, Inhibitor of NF-κB; IKK, IκB Kinase; N-terminal, amino-terminal; C-terminal, carboxy-terminal; ORF, open reading frame; RT, reverse transcriptase; β-gal, β-galactosidase; His, histidine; aa, amino acid; HA-tagged, hemagglutinin epitope tagged fusion construct; His-tagged, poly-histidine fusion construct; TBST, Tris buffered saline with 0.1% Tween-20; IMDM, Iscove's Modified Dulbecco's Media.

TRAF-3 gene products are signaling adaptor molecules required for lymphocytes to mediate T-dependent antibody responses in vivo. Previous work identified 8 splice-variant TRAF-3 mRNA species by RT-PCR that have the potential to encode novel isoforms, seven of which induce NF-κB activation when over-expressed in 293 cells. Here, their expression was characterized by RNAse protection assay, which showed the T cell line Jurkat D1.1 and the B cell lines BJAB, Daudi, and Raji each expressed mRNA encoding TRAF-3 splice-variants in approximately the same rank order (from highest to lowest); TRAF-3 Δ103aa, Δ83aa, full-length, Δ25aa, Δ52aa, Δ56aa, Δ27aa, and Δ221aa mRNA. The TRAF-3 Δ130aa mRNA was not detectable in any of the cell lines examined. The functional effect of over-expressing each TRAF-3 splice-variant on NF-κB activation was studied in the TRAF-5-responsive B cell line, BJAB. Of the seven TRAF-3 splice-variant isoforms that induce NF-κB activation in 293 cells, only TRAF-3 Δ27aa, Δ103aa, or Δ130aa induce NF-κB activation in BJAB cells. Together, these data indicate that a number of TRAF-3 splice-variant mRNAs are expressed and function in B and T lymphoma lines, which suggests that certain TRAF-3 splice-variant isoforms may participate in mediating the known functions of the TRAF-3 gene in lymphocytes.

TRAF-3 gene products are signaling adaptor molecules that interact with the cytoplasmic tails of CD40 (1-4) and other Tumor Necrosis Factor-Receptor (TNF-R) family members (e.g. LTβ-R, CD30, CD27, OX40) (4-9). Eight TRAF-3 mRNA splice-variants were identified by RT-PCR that have the potential to encode isoforms with altered Zn finger domains (3; 10-12). In addition, over-expression of seven of the eight putative TRAF-3 splice-variant isoforms induces NF-κB activation in 293 cells (12). However neither the expression nor function of mRNA for these splice-variants has been studied systematically in lymphoid cells. Therefore, the present study addressed whether TRAF-3 mRNA splice-variants are expressed or induce NF-κB activation in B or T lymphoma cells.

The signaling function of TRAF proteins has been inferred largely from the effects of transient over-expression of TRAFs in 293 cells, an adenovirus transformed human kidney epithelial cell line (13). By these criteria, TRAF-2 and TRAF-5 were thought to be NF-κB activating and TRAF-3 to be inhibitory (13-15). However, subsequent work demonstrated that neither TRAF-3 nor TRAF-2 could be considered to be genes that encode simply inhibitory or stimulatory functions, since over-expression of seven TRAF-3 splice-variant isoforms, individually, induces NF-κB activation in 293 cells (12) and a murine TRAF-2A splice-variant isoform (which might be considered a “full-length TRAF-2”) inhibits NF-κB activation (16). Although the TRAF-2A isoform is restricted to the murine and not the human gene (17), these findings suggest that each TRAF-3 or TRAF-2 splice-variant isoforms may have distinct functional potentials. In addition, it is unclear whether functional effects in 293 cells corresponds to functional potential in lymphocytes, where TRAF-3 is predominantly expressed (10;11).

The present study analyzed the expression of full-length TRAF-3 and eight characterized mRNA splice-variants by RNAse protection assays in a panel of B and T lymphoma lines. Seven of 8 TRAF-3 mRNA splice-variants, as well as full-length TRAF-3, were expressed by the TRAF-3+lymphoma cells and each variant contributed to the total TRAF-3 mRNA in a similar rank order in the different lymphomas. In addition, none of the cell lines expressed detectable levels of mRNA encoding TRAF-3 Δ130aa. The functional effect of over-expressing each TRAF-3 mRNA splice-variant isoform on NF-κB activation was studied in the B cell line, BJAB. Of the seven TRAF-3 splice-variant isoforms that induce NF-κB activation in 293 cells, only TRAF-3 Δ27aa, Δ103aa, or Δ130aa induced NF-κB activation in BJAB cells. Together, these data indicate that a number of TRAF-3 splice-variant mRNAs are expressed and function in B and T lymphoma lines.

### DETAILED DESCRIPTION OF THE INVENTION

Materials and methods

Plasmid Constructs

DNA fragments encoding subdomains of full-length TRAF-3 (FL) and 8 TRAF-3 splice-variant isoforms (Δ25, Δ27, Δ52, Δ56, Δ83, Δ103, Δ130, Δ221) which had been generated by RT-PCR amplification (12), were isolated by restriction digestion with EcoR I and BamH I (Roche Molecular Biochemicals, Indianapolis, Ind.) and ligated into pBluescript II SK+ (Stratagene, La Jolla, Calif.) for use in in vitro transcription (IVT) reactions. These constructs are termed; pIVT-FL, pIVT-Δ25, pIVT-Δ27, pIVT-Δ52, pIVT-Δ56, pIVT-Δ83, pIVT-Δ103, pIVT-Δ130, and pIVT-Δ221.

To generate an internal control, TRAF-3 sense RNA generating construct, PCR was used to amplify an exon 11 encoded fragment, TRAF-3 nt 1190-1246 (numbering from TRAF-3 (CRAF1) GenBank accession number U21092), using the primers Exon 11.for (5′-ATCGAATTCGGTACCAGCCAAGCAGAGAAACTGAAG-3′) and Coiled.rev (5′-CGCGGATCCAAGCTTCTAGTTCTGCCGGAAGGGCCGGATC-3′). This reaction utilized the Expand High Fidelity PCR System (Roche Molecular Biochemicals) and PCR conditions; 2 min. 94° C. (1 cycle); 15 sec. 94° C., 30 sec. 55° C., 45 sec. 72° C. (10 cycles); 15 sec. 94° C., 30 sec. 55° C., 1 min. 72° C. (10 cycles); 7 min. 72° C. (1 cycle). The PCR product was digested with EcoR I and BamH I and ligated into pBluescript II SK+ yielding pIVT-Exon-11/Sense.

Mammalian expression constructs in pCEP4 (Invitrogen, Carlsbad, Calif.) encoding TRAF-2, full-length TRAF-3, and TRAF-3 splice-variant isoforms, as well as the NF-κB dependent luciferase reporter PRDIIx4 Luc, have been described previously (12). The β-galactosidase (β-gal) open reading frame (ORF) was amplified using gene specific primers from pEBVHis LacZ (Invitrogen), digested with Xba I and BamH I (Roche Molecular Biochemicals) and ligated into the hemagglutinin (HA)-epitope tag expression vector pCGN (pCGN/LacZ). TRAF-5 was amplified by RT-PCR from Jurkat D1.1 mRNA with gene specific primers, ligated into pCR2.1 (Invitrogen), and sequenced. The TRAF-5 open reading frame was released from pCR2.1 by digestion with Sma I and BamH I (Roche Molecular Biochemicals) and ligated into corresponding sites in pCGN (pCGN/TRAF-5). Fragments encoding a portion of the CMV promoter and the HA tagged β-gal ORF or a portion of the CMV promoter and the HA tagged TRAF-5 ORF were released from pCGN by digestion with SnaB I and BamH I and ligated into the corresponding sites in pCEP4 (pCEP4/HA-LacZ and pCEP4/HA-TRAF-5). pTRI-GAPDH Human Antisense Control Template encoding nt 366-680 of the human GAPDH cDNA was obtained from Ambion (Austin, Tex.).

In Vitro Transcription

The pIVT-Δ221 construct was linearized with EcoR I. All other pIVT-TRAF-3 probe constructs were linearized with BstAP I (New England Biolabs, Beverly, Mass.), then blunted by addition of 3 units of T4 DNA Polymerase (New England Biolabs) in the presence of dNTPs (Roche Molecular Biochemicals) at a final concentration of 100 μM each, followed by incubation at 12° C. for 20 min. Samples were separated by agarose gel electrophoresis and gel purified using the Qiaquick Gel Extraction Kit (Qiagen, Valencia, Calif.) according to the manufacturer's instructions. Samples were ethanol precipitated and resuspended in distilled water at a concentration of approximately 0.5 μg/μl.

In vitro transcription (IVT) reactions were performed using the Riboprobe in vitro Transcription Systems kit (Promega, Madison, Wis.) according to the manufacturer's instructions using 50 μCi of α-³²P CTP (NEN Life Science Products, Boston, Mass.) per reaction. Each of the pIVT TRAF-3 constructs were transcribed using approximately 1 μg of linearized DNA and T3 RNA polymerase to generate anti-sense radiolabeled probes. pTRI-GAPDH was transcribed using 500 ng of linearized DNA and T7 RNA polymerase. Probes were resolved by denaturing 6% PAGE, excised from the gel following autoradiography, and extracted at 37° C. for 2 h in buffer containing 2 M ammonium acetate (Sigma, St. Louis, Mo.), 1% SDS (Roche Molecular Biochemicals), and 25 μg/ml yeast tRNA (Sigma). Extracted probes were ethanol precipitated, washed with cold 70% ethanol, dried, and resuspended in 50 μl of DEPC (Sigma) treated distilled water. The radioactivity of a 1 μl aliquot of each probe was measured in a Bioscan QC 2000 (Bioscan, Washington, D.C.), and probes were diluted with DEPC treated distilled water to 25,000 cpm/μl.

A non-radioactive control sense RNA was synthesized from the pIVT-Exon-11/Sense construct using T7 RNA polymerase. Diluted aliquots of the reaction product were stored at −80° C. until use. Control hybridization reactions were performed on serial dilutions of the sense RNA in order to determine the amount to add to samples as an internal hybridization control during experimental hybridizations.

RNAse Protection Assays

Total RNA was isolated from the B cell lines Ramos CC, BJAB, Daudi, and Raji, and the T cell line Jurkat D1.1 using the RNEasy Mini Kit (Qiagen) according to the manufacturer's instructions. The concentration of RNA was measured by the 260 nm Absorbance in a Beckmann DU-65 Spectrophotometer (Beckmann Instruments, Palo Alto, Calif.) and stored in distilled water at −80° C. until use.

Three hundred μg aliquots of yeast tRNA or total RNA from the indicated cell lines were mixed with 2 μl of a 1:10000 dilution of the TRAF-3 transcript from pIVT-Exon 11/Sense, 0.15 volumes of 2M sodium acetate, and ethanol precipitated. Pellets were washed with cold 70% ethanol, then dried for 5 min at room temperature. Pellets were resuspended in 300 μl of Hybridization Buffer containing 80% Formamide, 40 mM Pipes, 0.4 M sodium chloride (all Fisher Scientific, Pittsburgh, Pa.), and 1 mM EDTA (Digene Diagnostics, Beltsville, Md.) and heated briefly at 80° C. to facilitate resuspension. Hybridization reactions containing 30 μg of resuspended RNA and 2 μl (50,000 cpm) of probe were heated 5 min at 80° C. then immediately transferred to 45° C. and hybridized overnight. Three hundred μl of RNAse digestion mixture containing 10 mM Tris pH 7.5, 300 mM sodium chloride, 5 mM EDTA, and 40 μg of RNAse A (Roche Molecular Biochemicals) and 1500 U RNAse T1 (Roche Molecular Biochemicals) per ml was added per reaction and samples were digested at 37° C. for 60 min. RNAses were inactivated by addition of 20 μl of 10% SDS (Roche Molecular Biochemicals), 160 μg of Proteinase K (Roche Molecular Biochemicals), and incubation at 37° C. for 30 min. Samples were extracted with phenol:chloroform. Supernatants were ethanol precipitated with 20 μg of yeast tRNA as a carrier. Pellets were rinsed with 70% ethanol, dried for 5 min, and resuspended in 5 μl of RNA loading buffer containing 80% formamide, 1 mM EDTA pH 8.0, 0.1% Bromphenol Blue (Malinckrodt, Phillipsburg, N.J.), and 0.1% Xylene Cyanol (International Biotechnologies, New Haven, Conn.). Samples were heated to 80° C. for 5 min and loaded on prewarmed 6% denaturing polyacrylamide sequencing gels. Gels were run at 60 W for 130 min. then dried and exposed to Biomax MS film (Kodak, Rochester, N.Y.) or Phosphorimager screens (Molecular Dynamics, Sunnyvale, Calif.). Phosphorimager screens were scanned on 445SI and Storm 820 scanners (Molecular Dynamics).

Image analysis was performed using ImageQuant software version 1.2 for the Macintosh (Molecular Dynamics). Ten pixel wide polylines were drawn over sample lanes. The Peak Finder function was used to identify primary fragment signal peaks and eliminate non-specific peaks present in the yeast tRNA control. Background signal intensities observed for Ramos CC samples were measured at the corresponding positions to peaks observed for Jurkat D1.1 samples on the same gel. Signal intensities for primary fragments protected by the TRAF-3 Δ130 probe (from pIVT-Δ130) were measured at a position corresponding to the expected size of protected fragment, which is not visible to the eye. Peak areas were corrected for fragment size and normalized to the intensity of the 72 nt peak corresponding to protection of probe by the artificial TRAF-3 Exon 11 sense transcript, from pIVT-exon-11/sense. Finally, signals for each cell line were normalized to the level of GAPDH expression measured in parallel reactions to allow comparison of signal intensities between different cell lines. The data in FIG. 2 are scaled to a metric in which the signal for full length TRAF-3 mRNA in Jurkat D1.1 has a value of 100. The data in Table 2 represent the percentage contributions of individual TRAF-3 splice-variant mRNAs to total TRAF-3 mRNA. These percentages were obtained by dividing the variant expression in each individual cell line (background subtracted) and dividing by the total TRAF-3 expression for all splice-variants (background subtracted) in that cell line.

Cell Culture

Ramos CC and Jurkat D1.1 cells were cultured in IMDM (Life Technologies) supplemented with 10% Fetal Bovine Serum (Summit Biotechnologies) and 50 U/ml Penicillin, 50 μg/ml Streptomycin (Sigma). BJAB cells were kindly provided by Dr. Ricardo Dalla Fevera, Columbia University. Daudi, and Raji cells were purchased from ATCC (Rockville, Md.). BJAB, Daudi, and Raji cells were cultured in RPMI 1640 (Cellgro) supplemented with 17.8 mM sodium bicarbonate (Fisher Scientific), 10 mM Hepes (Fisher Scientific), 1 mM sodium pyruvate (Sigma), 2 mM glutamate (Life Technologies), 25 mM glucose (Fisher Scientific), and 50 U/ml Penicillin, 50 μg/ml Streptomycin (Sigma). Cells were cultured at 37° C. and 5% CO₂ in humidified incubators.

Transient Transfections and Luciferase Assays

BJAB cells were harvested and resuspended in serum free RPMI at 5 million per 300 μl. A 300 μl aliquot was mixed with 10 μg of the indicated expression vector, 1 μg of PRDIIx4 Luc, and 250 ng of pRLtk. Cells were transferred to 0.4 cm Gene Pulser Cuvettes (BioRad, Hercules, Calif.) and electroporated at 270 V and 975 μF using a Gene Pulser II with a Pulse Controller Plus and Capacitance Extender Plus (BioRad). Cells were harvested following electroporation by adding 1 ml of normal culture medium to the cuvette, then washing with an additional 1.7 ml normal culture medium and pooling the sample in one well of a six well tissue culture dish. Cells were cultured for approximately 36 hours, then harvested, washed with 1×PBS, and pelleted for measurement of luciferase activity. Samples were lysed in 200 μl of 1× Passive Lysis Buffer and assayed using the Dual Luciferase Assay Kit (Promega, Madison, Wis.) according to the manufacturer's instructions. Firefly luciferase reporter levels were normalized for transfection efficiency with the internal Renilla luciferase control. Values were scaled to the level of Firefly luciferase activity observed in samples transfected with the LacZ control. Data shown are representative of 3 independent experiments and error bars represent the standard deviation of measurements from triplicate samples.

Results

RNAse Protection Analysis of TRAF-3 Splice-Variant Isoform Expression.

In addition to full-length TRAF-3, eight alternatively spliced TRAF-3 mRNA species were recently identified by RT-PCR (12). In order to determine whether these TRAF-3 mRNA splice-variants are expressed in lymphoma cells, RNAse protection assays were performed using nine unique probes that were designed to protect distinct fragments when hybridized to the corresponding complementary splice-variant mRNA (Table 1 and FIG. 1A). As a positive control, a short, artificial sense TRAF-3 RNA (from exon 11) was added to each hybridization reaction to allow comparison of the signal intensities detected by the different probes. As negative controls, tRNA from yeast and RNA from the TRAF-3⁻ lymphoma line Ramos CC were examined in parallel (10;11). As expected, each probe protected the internal positive control (FIGS. 1C and 1D) and neither yeast tRNA nor Ramos CC mRNA protected significant amounts of any probe (FIG. 1C), which showed that the RNAse protection assay was specific for TRAF-3 mRNA species.

In the RNAse protection assay, RNA from the T cell line Jurkat D1.1 and the B cell lines BJAB, Daudi, and Raji protected the predicted primary (or largest) fragments of each probe (listed in Table 1) corresponding to TRAF-3 full-length, Δ25aa, Δ27aa, Δ52aa, Δ56aa, Δ83aa, Δ103aa, and Δ221aa mRNA splice-variants (indicated by * in FIGS. 1C and 1D). One unexpected finding was that none of the RNA samples protected the predicted fragment from the TRAF-3 Δ130aa probe (expected position indicated by * in FIGS. 1C and 1D). This finding did not appear to result from degradation of the TRAF-3 Δ130aa probe, since this probe migrated appropriately and protected the internal control TRAF-3 RNA at a level similar to the other probes. Thus the inability of any RNA sample to protect the expected fragment for TRAF-3 Δ130aa indicates that the TRAF-3 Δ130aa splice-variant is expressed below the limits of sensitivity for the assay. Together, these data indicate that a number of TRAF-3 mRNA species, including full-length and seven of eight characterized splice-variants, are expressed in the lymphoma lines Jurkat D1.1, BJAB, Daudi, and Raji.

In addition to the primary protected fragment of each probe to its corresponding mRNA splice-variant, secondary bands were predicted to occur from hybridization of each probe to other splice-variant mRNA species (e.g., secondary bands predicted to arise from protection of the full-length TRAF-3 probe are listed in FIG. 1A). Most of these predicted secondary bands were observed in each TRAF-3 expressing cell line. As an example, a 304 nt band corresponding to protection of mRNA encoded by exons 8 through 10 was detected by the probes representing TRAF-3 full length, Δ25, Δ27, and Δ52 but not by other probes (FIGS. 1C and 1D). Therefore, the protection of secondary bands confirmed and extended the analysis based on primary bands. However, an unexpected band of approximately 165 nt was detected by six probes (indicated by † in FIGS. 1C and 1D), which suggests that an additional TRAF-3 splice-variant mRNA exists that contains sequence from exons 10 and 11 (sequence common to all of the probes with a 165 nt protected fragment). Together, these data strongly suggest that, except for one additional band that seems to represent a single novel splice variant, the eight cloned cDNAs account for all of the alternatively spliced TRAF-3 mRNA species in these lymphoma lines that hybridize to these nine probes.

The intensities of the protected fragments in FIGS. 1C and 1D were measured using a PhosphorImager and normalized to GAPDH expression levels (FIG. 1B) to allow comparison of TRAF-3 splice-variant mRNA expression between cell lines (FIG. 2). The level of TRAF-3 mRNA varied approximately four-fold between the cell lines and was highest in Jurkat D1.1 and Raji (relative to GAPDH), and relatively lower in BJAB and Daudi (FIG. 2). The relative contribution of individual splice-variants to the total amount of TRAF-3 mRNA within each cell line was similar (Table 2). The splice-variants contributing most to TRAF-3 mRNA expression are Δ103aa and Δ83aa, which together comprise more than 40% the TRAF-3 mRNA in any of the lines (Table 2). Full-length TRAF-3 and Δ25aa are approximately twice as abundant as the Δ52aa and Δ56aa splice-variants. The splice-variants Δ27aa and Δ221aa each comprise less than 8% of the TRAF-3 mRNA in any cell line. TRAF-3Δ130aa was undetectable above background signals from yeast tRNA or Ramos CC, even by phosphorimaging analysis of the gel position estimated to coincide with the primary probe fragment that would be protected by any TRAF-3Δ130aa mRNA (FIG. 2 and Table 2). Thus, these data indicate that relative contributions made by individual splice-variants to the total amount of TRAF-3 mRNA appear to be similar in different lymphoma cell lines.

Effects of Over-Expressing TRAF-3 Splice Deletion Variants on NF-κB Activation in BJAB.

The next experiments addressed whether the TRAF-3 splice deletion isoforms are capable of inducing NF-κB activation in the B cell line BJAB. BJAB cells were selected for functional studies due to their relatively efficient transfection by electroporation and their low background of NF-κB reporter gene expression. The effects of over-expressing TRAF-3 splice-variants or full-length TRAF-3 were compared with those of over-expressing TRAF-2 and TRAF-5, which are known to induce NF-κB activation in 293 cells (13-15). In these experiments, the background NF-κB reporter gene activity was determined from parallel samples transiently transfected with β-galactosidase (indicated by LacZ in FIG. 3). As expected, over-expression of TRAF-5 induces NF-κB reporter gene expression, whereas full-length TRAF-3 or TRAF-3 Δ221aa are inactive (FIG. 3). In addition, over-expression of TRAF-3 Δ27aa, Δ103aa, or Δ130aa induces NF-κB activation in BJAB cells, 2-fold, 4-fold, and 11-fold, respectively (FIG. 3), similar to their ability to induce NF-κB activation in 293 cells (12). Surprisingly, over-expression of TRAF-3 Δ25aa, Δ52aa, Δ56aa, Δ83aa, or TRAF-2 failed to induce NF-κB activation, despite the ability of these constructs to induce strong activation of NF-κB in 293 cells (12). Together, these data suggest that only certain TRAF-3 splice-variant isoforms induce NF-κB activation in B lymphoma cells.

Discussion

Previous work identified eight TRAF-3 mRNA splice-deletion variants by RT-PCR (12). In the present study, RNAse protection analysis established that, in addition to full-length TRAF-3, seven of these eight alternatively spliced TRAF-3 mRNA species are expressed in the lymphoma cell lines BJAB, Daudi, Raji, and Jurkat D1.1, and that each species contributed a similar proportion to the total TRAF-3 mRNA level in the cell lines. Although seven of the TRAF-3 splice-variants had been previously shown to induce NF-κB activation when over-expressed in 293 cells, only three TRAF-3 splice-variant isoforms (Δ27aa, Δ103aa, and Δ130aa) induced NF-κB activation in BJAB cells.

The present study confirmed by RNAse protection analysis that seven of eight mRNA splice-variant species previously cloned by RT-PCR are expressed in lymphoma lines, but also showed that TRAF-3 Δ221aa mRNA was expressed at very low levels and, even by phosphorimaging analysis, TRAF-3 Δ130aa did not appear to be expressed above background signals. The RNAse protection assay provides a more accurate measure of steady state mRNA levels than RT-PCR because RNAse protection does not rely on amplification. Therefore, the disproportionate intensity of RT-PCR bands for Δ221aa and Δ130aa may represent preferential amplification of these relatively small products during PCR (12). In addition, the finding by RNAse protection analysis that Δ221aa is expressed at very low levels and Δ130aa mRNA is undetectable, suggests that peptide bands previously associated with these mRNA species may represent proteolytic TRAF-3 fragments or proteins encoded by additional, uncharacterized TRAF-3 splice-variant mRNAs (12).

Although this study examined the expression and signaling properties of full-length TRAF-3 and eight splice-variant mRNAs, data exist in support of additional TRAF-3 splice-variants. For example, a cDNA clone has been described that is predicted to encode a TRAF-3 isoform lacking the N-terminal RING and Zn fingers due to translation initiation at an internal methionine within the coiled-coil domain (4). In addition, the RNAse protection analysis described in this work revealed a TRAF-3 specific band of approximately 165 nt that did not correspond to any of the previously cloned splice-variant mRNAs (indicated by t in FIGS. 1C and D). Since an apparently identical 165 nt band was protected by the probes representing full-length, Δ25aa, Δ27aa, Δ52aa, Δ56aa, and Δ83aa splice-variants, this band may represent a single, novel splice-variant mRNA that contains sequences from exon 10 and 11. Although the identity of the protected fragment is not certain, the data are consistent with an mRNA the results from a cryptic splice acceptor encoded in exon 10. It is interesting to note that splicing of exon 3 to this putative splice acceptor site could maintain an open reading frame and encode a polypeptide that lacks part of the RING finger, all 5 Zn fingers, and a large proportion of the coiled-coil domain of TRAF-3. A more complete understanding of TRAF-3 signaling will require further studies of TRAF-3 mRNA splicing and the resulting expression of TRAF-3 splice-variant isoforms.

The levels of TRAF-3 mRNA expression were found to vary approximately 4-fold between the four TRAF-3+lymphoma lines and TRAF-3 mRNA was undetectable in Ramos CC. It is interesting to consider the possibility that TRAF-3 expression varies in these different lymphoma lines in a manner that may model its regulation in normal lymphocytes. In support of this idea, Jurkat D1.1, which has the phenotype of an activated T cell (18), expresses relatively high levels of TRAF-3 mRNA, which may be analogous to the finding that CD3 cross-linking induces TRAF-3 expression in PBL (10).

Despite the variation in levels of TRAF-3 mRNA, the four TRAF-3⁺ cell lines were found to express TRAF-3 splice-variant mRNAs in a similar rank order. To the extent that lymphoma cell lines model features of different cell lineages or stages of differentiation, these data suggest that alternative splicing to each mRNA variant may occur in a fixed proportion in lymphoid cells and may not be regulated. However, in addition to lymphoid cells, TRAF-3 mRNA is expressed by a wide variety of other tissues and cell types (2;10;11). Therefore, it will be of interest to determine whether or not TRAF-3 expressing cells of different lineages or stages of differentiation contain the same relative proportions of TRAF-3 mRNA splice-variants observed in lymphoma lines. Since each TRAF-3 splice-variant isoform may possess differing signaling abilities, changes in the either the absolute amount or relative proportion of a particular splice-variant in a cell may affect the threshold of receptor stimulation required to initiate downstream signaling.

The finding that TRAF-3 Δ130aa isoform induced a high level of NF-κB activation after over-expression in BJAB cells, is interesting in light of the fact that Δ130aa encoding mRNA was undetectable in any of the resting lymphoma lines. Since TRAF-3 Δ130aa encoding mRNA was cloned from Jurkat D1.1 (12), these data suggest that Δ130aa is expressed under certain circumstances, and may be both potent and closely regulated. It remains unclear how TRAF-3 over-expression is informative about the mechanism of receptor-induced signaling, and different pathways may exist for NF-κB activation and cJun N-terminal kinase signaling (19-21). One possibility for NF-κB mediated signaling studied here, is that receptor aggregation (by ligand) liberates TRAF-3 from receptor tails. In this scenario, high local concentrations of receptor tails and TRAF-3 homotrimers would favor TRAF-3 homotrimers either binding three or no receptor tails (22). The downstream events in TRAF-3 signaling may be cell type restricted since four TRAF-3 splice-variants which induce NF-κB activation in 293 cells (12) failed to induce NF-κB activation in BJAB cells. The basis of this cell-type restricted signaling are not yet understood, but may relate to differences in expression of TRAF binding kinases that stimulate the IKK complex, such as NIK, MEKK1, GCK, and GCKR (23-29). TRAF-2 signaling also appeared to be cell-type restricted since over-expression of TRAF-2 failed to induce NF-κB activation in BJAB whereas TRAF-5 was active.

The observation that certain TRAF-3 splice-variants and TRAF-5 share the ability to induce NF-κB activation in BJAB cells is interesting in light of recent reports that TRAF-3 and TRAF-5 may physically interact with each other and that such interactions may be required to recruit TRAF-5 to CD40 cytoplasmic tails (30;31). An additional similarity between activating TRAF-3 splice-variants and TRAF-5 is that they are thought to functionally interact with full-length TRAF-3, since their ability to induce NF-κB activation is augmented by co-expression with full-length TRAF-3 (12;31). These data suggest that TRAF-3 splice-variants or TRAF-5 might function together with full-length TRAF-3 in receptor-triggered signaling. Although the physiological roles for TRAF-3 splice-variants are not completely understood, redundant functional characteristics of TRAF-3 splice-variants and TRAF-5 may account for the preservation of CD40 triggered NF-κB activation in TRAF-5 deficient B cells (32). In addition, if TRAF-5 participation in CD40 signaling depends on TRAF-3 mediated recruitment, the more severe phenotype of the TRAF-3 deficient mouse with respect to T-dependent antibody responses might arise from a functional disruption of both TRAF-3 and TRAF-5 dependent CD40 signaling (32;33).

The finding that certain TRAF-3 mRNA splice-variants are expressed and are capable of inducing NF-κB activation in lymphoma lines, suggests that TRAF-3 splice-variant isoforms may functionally participate in transduction of positive signals from CD40 into the nucleus in lymphocytes. Thus, the biology of TRAF-3 appears to be intimately associated with alternative splicing and the function of splice products. Since abnormal mRNA splicing accounts for a substantial proportion of genetic diseases (34), it will be of interest to determine whether abnormalities in TRAF-3 splicing might underlie defects in CD40 signaling in certain individuals with Hyper-IgM syndrome and normal CD154 and normal activation-induced cytidine deaminase (35-37).

TABLE 1
Splice-Variant Specific Anti-sense RNA Probes and Expected Sizes
for Protected Fragments from RNAse Protection Analysis

| Splice | Exons Omitted | | Protected |
|---|---|---|---|
| Isoform | by Splicing | Probe (nt) | Fragment (nt) |
| FL | — | 555 | 494 |
| Δ25 | 8 | 480 | 419 |
| Δ27 | 7 | 474 | 413 |
| Δ52 | 7.8 | 399 | 338 |
| Δ56 | 8.9 | 387 | 326 |
| Δ83 | 7-9 | 306 | 251 |
| Δ103 | 8-10 | 246 | 185 |
| Δ130 | 7-10 | 165 | 110 |
| Δ221 | 5-10 | 304 | 232 |

TABLE 2
Contribution of Individual TRAF-3 Splice-Variant
mRNAs to Total TRAF-3 mRNA in Lymphoma Cell Lines (%)

| Cell | | | | | | | | | |
|---|---|---|---|---|---|---|---|---|---|
| Line | FL | Δ25 | Δ27 | Δ52 | Δ56 | Δ83 | Δ103 | Δ130 | Δ221 |
| D1.1 | 18.3 | 16.7 | 3.3 | 8.5 | 9.4 | 15.4 | 26.3 | 0.3 | 1.9 |
| BJAB | 11.2 | 2.3 | 2.5 | 6.7 | 6.3 | 27.5 | 43.5 | N.D. | N.D. |
| Daudi | 16.1 | 8.8 | 7.7 | 6.7 | 5.5 | 20.4 | 28.8 | N.D. | 6.0 |
| Raji | 11.7 | 10.6 | 1.7 | 7.3 | 10.5 | 21.0 | 34.2 | 0.3 | 2.7 |

N.D., not detectable above background observed for Ramos CC

### REFERENCES

(1) Hu H M, O'Rourke K, Boguski M S, Dixit V M. A novel RING finger protein interacts with the cytoplasmic domain of CD40. J Biol Chem 269:30069, 1994.
(2) Cheng, G, Cleary, A M, Ye, Z-S, Hong, D I, Lederman, S, and Baltimore, D. Involvement of CRAF1, a relative of TRAF, in CD40 signaling. Science 267: 1494, 1995.
(3) Sato T, Irie S, Reed J C. A novel member of the TRAF family of putative signal transducing proteins binds to the cytosolic domain of CD40. FEBS Letters 358:113, 1995.
(4) Mosialos G, Birkenbach M, Yalamanchili R, VanArsdale T, Ware C, Kieff E. The Epstein-Barr virus transforming protein LMP1 engages signaling proteins for the tumor necrosis factor receptor family. Cell. 80:389, 1995.
(5) Gedrich R W, Gilfillan M C, Duckett C S, Van Dongen J L, Thompson C B. CD30 contains two binding sites with different specificities for members of the tumor necrosis factor receptor-associated factor family of signal transducing proteins. J Biol Chem 271:12852, 1996.
(6) Boucher L M, Marengere L E, Lu Y, Thukral S, Mak T W. Binding sites of cytoplasmic effectors TRAF1, 2, and 3 on CD30 and other members of the TNF receptor superfamily. Biochem Biophys Res Commun 233:592, 1997.
(7) Vanarsdale T L, VanArsdale S L, Force W R, Walter B N, Mosialos G, Kieff E et al. Lymphotoxin-beta receptor signaling complex: role of tumor necrosis factor receptor-associated factor 3 recruitment in cell death and activation of nuclear factor KB. Proc Natl Acad Sci USA 94:2460, 1997.
(8) Arch R H, Thompson C B. 4-1BB and Ox40 are members of a tumor necrosis factor (TNF)—nerve growth factor receptor subfamily that bind TNF receptor-associated factors and activate nuclear factor KB. Mol Cell Biol 18:558, 1998.
(9) Kawamata S, Hori T, Imura A, Takaori-Kondo A, Uchiyama T. Activation of OX40 signal transduction pathways leads to tumor necrosis factor receptor-associated factor (TRAF) 2- and TRAF5-mediated NF-κB activation. J Biol. Chem. 273:5808, 1998.
(10) Krajewski, S., Zapata, J. M., Krajewska, M., VanArsdale, T., Shabaik, A., Gascoyne, R. D., and Reed, J. C. Immunohistochemical analysis of in vivo patterns of TRAF-3 expression, a member of the TNF receptor-associated factor family. J Immunol 159:5841, 1997.
(11) van Eyndhoven, W. G., Frank, D., Kalachikov, S., Cleary, A. M., Hong, D. I., Cho, E., Nasr, S., Perez, A. J., Mackus, W. J. M., Cayanis, E., Wellington, S., Fischer, S. G., Warburton, D., and Lederman, S. A single gene for human TRAF-3 at chromosome 14q32.3 encodes a variety of mRNA species by alternative polyadenylation, mRNA splicing and transcription initiation. Mol Immunol 35:1189, 1998.
(12) van Eyndhoven W G, Gamper C J, Cho E, Mackus W J M, Lederman S. TRAF-3 mRNA splice-deletion variants encode isoforms that induce NF-κB activation. Mol Imm. 36:647, 1999.
(13) Rothe M, Sarma V, Dixit V M, Goeddel D V. TRAF2-mediated activation of NF-κ-B by TNF Receptor 2 and CD40. Science 269:1424, 1995.
(14) Nakano H, Oshima H, Chung W, Williams-Abbott L, Ware C F, Yagita H et al. TRAF5, an activator of NF-kappaB and putative signal transducer for the lymphotoxin-β receptor. J Biol. Chem. 271:14661, 1996.
(15) Ishida T K, Tojo T, Aoki T, Kobayashi N, Ohishi T, Watanabe T et al. TRAF5, a novel tumor necrosis factor receptor-associated factor family protein, mediates CD40 signaling. Proc Natl Acad Sci USA 93:9437, 1996.
(16) Brink, R. and Lodish, H. F. Tumor necrosis factor receptor (TNRF)-associated factor 2A (TRAF2A), a TRAF2 splice variant with an extended RING finger domain that inhibits TNFR2-mediated NF-κB activation. J Biol Chem 273:4129, 1998.
(17) Grech A, Quinn R, Srinivasan D, Badoux X, Brink R. Complete structural characterisation of the mammalian and Drosophila TRAF genes: implications for TRAF evolution and the role of RING finger splice variants. Mol Immunol 37:721, 2000.
(18) Lederman S, Yellin M J, Krichevsky A, Belko J, Lee J J, Chess L. Identification of a novel surface protein on activated CD4+ T cells that induces contact dependent B cell differentiation (help). J Exp Med. 175:1091, 1992.
(19) Dadgostar H, Cheng G. Membrane localization of TRAF 3 enables JNK activation. J Biol Chem 275:2539, 2000.
(20) Hostager B S, Catlett I M, Bishop G A. Recruitment of CD40 and tumor necrosis factor receptor-associated factors 2 and 3 to membrane microdomains during CD40 signaling. J Biol Chem 275:15392, 2000.
(21) Dadgostar H, Cheng G. An intact zinc ring finger is required for tumor necrosis factor receptor-associated factor-mediated nuclear factor-KB activation but is dispensable for c-Jun N-terminal kinase signaling. J Biol Chem 273:24775, 1998.
(22) Pullen S S, Labadia M E, Ingraham R H, McWhirter S M, Everdeen D S, Alber T et al. High-affinity interactions of tumor necrosis factor receptor-associated factors (TRAFs) and CD40 require TRAF trimerization and CD40 multimerization. Biochemistry 38:10168, 1999.
(23) Malinin N L, Boldin M P, Kovalenko A V, Wallach D. MAP3K-related kinase involved in NF-κB induction by TNF, CD95 and IL-1. Nature 385:540, 1997.
(24) Yin L, Wu L, Wesche H, Arthur C D, White J M, Goeddel D V et al. Defective lymphotoxin-beta receptor-induced NF-κB transcriptional activity in NIK-deficient mice. Science 291:2162, 2001.
(25) Song H Y, Regnier C H, Kirschning C J, Goeddel D V, Rothe M. Tumor necrosis factor (TNF)-mediated kinase cascades: bifurcation of nuclear factor-KB and c-jun N-terminal kinase (JNK/SAPK) pathways at TNF receptor-associated factor 2. Proc Natl Acad Sci U S A 94:9792, 1997.
(26) Baud V, Liu Z G, Bennett B, Suzuki N, Xia Y, Karin M. Signaling by proinflammatory cytokines: oligomerization of TRAF2 and TRAF6 is sufficient for JNK and IKK activation and target gene induction via an amino-terminal effector domain. Genes Dev 13:1297, 1999.
(27) Yuasa T, Ohno S, Kehrl J H, Kyriakis J M. Tumor necrosis factor signaling to stress-activated protein kinase (SAPK)/Jun NH2-terminal kinase (JNK) and p38. Germinal center kinase couples TRAF2 to mitogen-activated protein kinase/ERK kinase kinase 1 and SAPK while receptor interacting protein associates with a mitogen-activated protein kinase kinase kinase upstream of MKK6 and p38. J Biol Chem 273:22681, 1998.
(28) Shi C S, Kehrl J H. Activation of stress-activated protein kinase/c-Jun N-terminal kinase, but not NF-κB, by the tumor necrosis factor (TNF) receptor 1 through a TNF receptor-associated factor 2- and germinal center kinase related-dependent pathway. J Biol Chem 272:32102, 1997.
(29) Chin A I, Shu J, Shan S C, Yao Z, Kehrl J H, Cheng G. TANK Potentiates Tumor Necrosis Factor Receptor-Associated Factor-Mediated c-Jun N-Terminal Kinase/Stress-Activated Protein Kinase Activation through the Germinal Center Kinase Pathway. Mol Cell Biol 19:6665, 1999.
(30) Pullen S S, Miller H G, Everdeen D S, Dang T T A, Crute J J, Kehry M R. CD40-tumor necrosis factor receptor-associated factor (TRAF) interactions: regulation of CD40 signaling through multiple TRAF binding sites and TRAF hetero-oligomerization. Biochemistry 37:11836, 1998.
(31) Leo E, Welsh K, Matsuzawa S, Zapata J M, Kitada S, Mitchell R S et al. Differential requirements for tumor necrosis factor receptor-associated factor family proteins in CD40-mediated induction of NF-κB and Jun N-terminal kinase activation. J Biol Chem 274:22414, 1999.
(32) Nakano H, Sakon S, Koseki H, Takemori T, Tada K, Matsumoto M et al. Targeted disruption of Traf5 gene causes defects in CD40- and CD27-mediated lymphocyte activation. Proc Natl Acad Sci USA. 96:9803, 1999.
(33) Xu Y, Cheng G, Baltimore D. Targeted disruption of TRAF3 leads to postnatal lethality and defective T-dependent immune responses. Immunity 5:407, 1996.
(34) Cooper T A, Mattox W. The regulation of splice-site selection, and its role in human disease. Am J Hum Genet 61:259, 1997.
(35) Durandy A, Hivroz C, Mazerolles F, Schiff C, Bernard F, Jouanguy E et al. Abnormal CD40-mediated activation pathway in B lymphocytes from patients with hyper-IgM syndrome and normal CD40 ligand expression. J Immunol 158:2576, 1997.
(36) Revy P, Muto T, Levy Y, Geissmann F, Plebani A, Sanal O et al. Activation-induced cytidine deaminase (AID) deficiency causes the autosomal recessive form of the Hyper-IgM syndrome (HIGM2). Cell 102:565, 2000.
(37) Minegishi Y, Lavoie A, Cunningham-Rundles C, Bedard P M, Hebert J, Cote L et al. Mutations in activation-induced cytidine deaminase in patients with hyper IgM syndrome. Clin Immunol 97:203, 2000.

What is claimed is:

1. An isolated polypeptide having the amino acid sequence set forth in SEQ ID NO:1.

2. An isolated polypeptide having an amino acid sequence that is at least 80% identical over its entire length to the amino acid sequence set forth in SEQ ID NO:1.

3. A pharmaceutical composition comprising a polypeptide having the amino acid sequence set forth in SEQ ID NO:1 and a pharmaceutically acceptable carrier.

4. A pharmaceutical composition comprising a polypeptide having the amino acid sequence that is at least 80% identical over its entire length to the amino acid sequence set forth in SEQ ID NO:1.

5. A pharmaceutical composition according to claim 3 in a unit dosage form.

6. A pharmaceutical composition according to claim 5 comprising from about 10 mg to about 100 mg of the polypeptide set forth in SEQ ID NO:1.

7. A pharmaceutical composition according to claim 6 comprising from about 10 mg to about 50 mg of the polypeptide.

8. A pharmaceutical composition according to claim 7 comprising from about 10 mg to about 30 mg of the polypeptide.

9. A pharmaceutical composition according to claim 5 wherein the unit dossage form is in the form of a tablet or capsule.

10. A pharmaceutical composition according to claim 4 in a unit dosage form.

11. A pharmaceutical composition according to claim 10 comprising from about 10 mg to about 100 mg of the polypeptide.

12. A pharmaceutical composition according to claim 11 comprising from about 10 mg to about 50 mg of the polypeptide.

13. A pharmaceutical composition according to claim 12 comprising from about 10 mg to about 30 mg of the polypeptide.

14. A pharmaceutical composition according to claim 10 wherein the unit dossage form is in the form of a tablet or capsule.

15. A pharmaceutical composition according to claim 5 wherein the unit dossage form is in the form of an injectable solution which comprises from about 500 mg to about 5,000 mg per liter.

16. A pharmaceutical composition according to claim 15 in the form of an injectable solution which comprises from about 500 mg to about 5000 mg per liter of a polypeptide that is at least 80% identical over its entire length to the amino acid sequence set forth in SEQ ID NO:1.

17. A method of inhibiting translocation of NF-6B across a nuclear membrane comprising introducing into a mammalian cell a polypeptide having the amino acid sequence set forth in SEQ ID NO:1 in an amount effective to inhibit the translocation of NF-6B across a nuclear membrane in the cell.

18. A method of inhibiting translocation of NF-6B across a nuclear membrane comprising introducing into a mammalian cell a polypeptide having an amino acid sequence that is at least 80% identical over its entire length to the amino acid sequence set forth in SEQ ID NO:1 in an amount effective to inhibit the translocation of NF-6B across a nuclear membrane in the cell.

19. A method of inhibiting inflammation of tissue in a mammal comprising administering to a mammal in need thereof a pharmaceutical composition comprising an effective amount of an isolated polypeptide having the amino acid sequence set forth in SEQ ID NO:1 and a pharmaceutically acceptable carrier.

20. A method of inhibiting inflammation of tissue in a mammal comprising administering to a mammal in need thereof a pharmaceutical composition comprising an effective amount of an isolated polypeptide having an amino acid sequence that is at least 80% identical over its entire length to the amino acid sequence set forth in SEQ ID NO:1 and a pharmaceutically acceptable carrier.

21. A method according to claim 19 wherein the mammal is a human.

22. A method according to claim 20 wherein the mammal is a human.

23. An isolated polypeptide having the amino acid sequence set forth in SEQ ID NO:2.

24. An isolated polypeptide having an amino acid sequence that is at least 80% identical over its entire length to the amino acid sequence set forth in SEQ ID NO:2.

25. A pharmaceutical composition comprising a polypeptide having the amino acid sequence set forth in SEQ ID NO:2 and a pharmaceutically acceptable carrier.

26. A pharmaceutical composition comprising a polypeptide having the amino acid sequence that is at least 80% identical over its entire length to the amino acid sequence set forth in SEQ ID NO:2.

27. A pharmaceutical composition according to claim 25 in a unit dosage form.

28. A pharmaceutical composition according to claim 27 comprising from about 10 mg to about 100 mg of the polypeptide set forth in SEQ ID NO:2.

29. A pharmaceutical composition according to claim 28 comprising from about 10 mg to about 50 mg of the polypeptide.

30. A pharmaceutical composition according to claim 29 comprising from about 10 mg to about 30 mg of the polypeptide.

31. A pharmaceutical composition according to claim 27 in the form of a tablet or capsule.

32. A pharmaceutical composition comprising a polypeptide having the amino acid sequence that is at least 80% identical over its entire length to the amino acid sequence set forth in SEQ ID NO:2 and a pharmaceutically acceptable carrier.

33. A pharmaceutical composition according to claim 32 in a unit dosage form.

34. A pharmaceutical composition according to claim 32 comprising from about 10 mg to about 100 mg of the polypeptide.

35. A pharmaceutical composition according to claim 34 comprising from about 10 mg to about 50 mg of the polypeptide.

36. A pharmaceutical composition according to claim 35 comprising from about 10 mg to about 30 mg of the polypeptide.

37. A pharmaceutical composition according to claim 32 wherein the unit dossage form is in the form of a tablet or capsule.

38. A pharmaceutical composition according to claim 27 in the form of an injectable solution which comprises from about 500 mg to about 5,000 mg per liter.

39. A pharmaceutical composition according to claim 37 in the form of an injectable solution which comprises from about 500 mg to about 1000 mg liter of a polypeptide according to claim 18.

40. A method of enhancing the activation of NF-6B in a mammalian cell comprising introducing into a mammalian cell a polypeptide having the amino acid sequence set forth in SEQ ID NO:2 in an amount effective to enhance the activation of NF-6B.

41. A method of enhancing the activation of NF-6B in a mammalian cell comprising introducing into a mammalian cell a polypeptide having an amino acid sequence that is at least 80% identical over its entire length to the amino acid sequence set forth in SEQ ID AFT NO:2 in an amount effective to enhance the activation of NF-6B.

42. A method of enhancing an immune response in a mammal comprising administering to a mammal in need thereof an effective amount of a pharmaceutical composition comprising the polypeptide having the sequence set forth in SEQ ID NO:2 and a pharmaceutically acceptable carrier.

43. A method of enhancing an immune response in a mammal comprising administering to a mammal in need thereof an effective amount of a pharmaceutical composition comprising a polypeptide having an amino acid sequence according to claim 18 and a pharmaceutically acceptable carrier.

44. A method according to claim 43 wherein the subject is a mammal.

45. A method according to claim 44 wherein the mammal is a human.

46. A method of screening compounds for effect on an interaction between components of a biochemical system, comprising:
incubating components of a biochemical system, a test compound, and a polypeptide selected from the group consisting of a polypeptide having the amino acid sequence set forth in SEQ ID NO:1, a polypeptide having an amino acid sequence that is at least 80% identical over its entire length to a polypeptide having the amino acid sequence set forth in SEQ ID NO:1, a polypeptide having the amino acid sequence set forth in SEQ ID NO:2, and a polypeptide having an amino acid sequence that is at least 80% identical over its entire length to a polypeptide having the amino acid sequence set forth in SEQ ID NO:2; and
detecting an effect of a test compound on the components of the biochemical system.

47. A method of screening compounds according to claim 46 wherein the components of the biochemical system produce a detectable signal representative of a function of the biochemical system.

48. A method of screening compounds according to claim 47 wherein the components of the biochemical system comprise an indicator compound which interacts with at least one other component of the biochemical system to produce a detectable signal representative of a function of the biochemical system.

49. A method of screening compounds according to claim 47 wherein the components of the biochemical system comprise an enzyme and an indicator compound comprises a substrate for the enzyme, wherein action of the enzyme on the substrate produces a detectable signal.

50. A method of screening compounds according to claim 49 wherein components of the biochemical system comprise a receptor/ligand binding pair, wherein at least one of the receptor or ligand has a detectable signal associated therewith.

51. A method of screening compounds according to claim 47 wherein the components of the biochemical system comprise a receptor/ligand binding pair, wherein binding of the receptor to the ligand produces a detectable signal.

52. A method of screening compounds according to claim 47 wherein the components of the biochemical system comprise cells, the detecting step further comprises determining an effect of the test compound on a cell.

53. A method of screening compounds according to claim 50, wherein a cell are capable of producing a detectable signal corresponding to a cellular function, and wherein the detecting step further comprises detecting an effect of the test compound on the cellular function by detecting a level of the detectable signal.

54. A method of screening compounds according to claim 51, wherein the cellular function is activation of NF-6B.

55. A method of screening compounds according to claim 52, wherein the cellular function is translocation across a cellular membrane.
